SEQUENCE LISTING (11 sequences)

SEQ ID NO: 1 - DNA, 1635 bp, Artificial Sequence
  Synthetic sequence
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 2 - protein, 254 aa, Artificial Sequence
  Synthetic protein
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 3 - protein, 21 aa, Artificial Sequence
  Synthetic protein
MALPVTALLLPLALLLHAARP

SEQ ID NO: 4 - protein, 45 aa, Artificial Sequence
  Synthetic protein
TTTPAPRPPTPAPTIASQPLSLRPEACRPAAGGAVHTRGLDFACD

SEQ ID NO: 5 - protein, 27 aa, Artificial Sequence
  Synthetic protein
FWVLVVVGGVLACYSLLVTVAFIIFWV

SEQ ID NO: 6 - protein, 41 aa, Artificial Sequence
  Synthetic protein
RSKRSRLLHSDYMNMTPRRPGPTRKHYQPYAPPRDFAAYRS

SEQ ID NO: 7 - protein, 42 aa, Artificial Sequence
  Synthetic protein
KRGRKKLLYIFKQPFMRPVQTTQEEDGCSCRFPEEEEGGCEL

SEQ ID NO: 8 - protein, 113 aa, Artificial Sequence
  Synthetic protein
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 543 aa, Artificial Sequence
  Sythetic protein
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 249 aa, Artificial Sequence
  Synthetic protein
QVRLQGSGPSLVKPSQTLSLTCTVSGFSLTSNAVDWVRQAPGKVPEWLGFIRGGGSTFYNSALKSRLSITRDTSKSQVSLSLSSVTTEDTAVYYCARASCSGDIYTDTCGIDYWGPGLLVTVSSEGKSSGSGSESKVDQSALTQPSSVSRSLGQSVSITCSGSSSNVGAGNYVNWFRLIPGSAPKSLIYAATTRASGVPDRFSGSRSGNTATLTISSLQAEDEADYYCSSYDITAVNLFGSGTRLTVLG

SEQ ID NO: 11 - protein, 538 aa, Artificial Sequence
  Synthetic protein
MALPVTALLLPLALLLHAARPQVRLQGSGPSLVKPSQTLSLTCTVSGFSLTSNAVDWVRQAPGKVPEWLGFIRGGGSTFYNSALKSRLSITRDTSKSQVSLSLSSVTTEDTAVYYCARASCSGDIYTDTCGIDYWGPGLLVTVSSEGKSSGSGSESKVDQSALTQPSSVSRSLGQSVSITCSGSSSNVGAGNYVNWFRLIPGSAPKSLIYAATTRASGVPDRFSGSRSGNTATLTISSLQAEDEADYYCSSYDITAVNLFGSGTRLTVLGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2020/0306376 A1
(43) Pub. Date: Oct. 1, 2020

(54) COMPOSITIONS AND METHODS FOR TARGETED TUMOR IMMUNOTHERAPY

(71) Applicant: Dana-Farber Cancer Institute, Inc., Boston, MA (US)

(72) Inventors: Carl Novina, Newton, MA (US); Robert J. Distel, Framingham, MA (US); Steven Neier, Waltham, MA (US); Alberto Nobili, Brookline, MA (US)

(73) Assignee: Dana-Farber Cancer Institute, Inc., Boston, MA (US)

(21) Appl. No.: 16/606,619

(22) PCT Filed: Apr. 25, 2018
(86) PCT No.: PCT/US2018/029424

§ 371 Date: Oct. 18, 2019

Related U.S. Application Data
(60) Provisional application No. 62/489,913, filed on Apr. 25, 2017.

(51) Int. Cl. A61K 41/00; A61K 47/68; A61K 35/17; A61K 9/00; C07K 14/705; C07K 14/725; C07K 16/18; C07K 16/44; C07D 493/10; C07C 235/66

(52) U.S. Cl. CPC A61K 41/0042; A61K 2039/505; A61K 35/17; A61K 9/0029; C07K 14/70517; C07K 14/70521; C07K 14/70578; C07K 14/7051; C07K 16/18; C07K 16/44; C07D 493/10; C07C 235/66; C07K 2319/02; C07K 2319/03; C07K 2319/30; C07K 2319/33; A61K 47/6851

(57) ABSTRACT

The present invention provides universal immunotherapy compositions useful for targeted treatment of cancers. The present invention utilizes in its various aspects, bifunctional compounds or complexes that contain at least two domains. One domain, referred to herein as a targeting moiety, binds an antigen on the surface of a tumor cell. The other domain, referred to herein as a pro-antigen, is designed to be inert to normal (non-diseased) cells and tissues, and to become activated (or “unmasked” or “unaged”) only upon exposure to light of an appropriate wavelength.

### RELATED APPLICATIONS

This application is a national stage application, filed under 35 U.S.C. §371, of International Application No. PCT/US2018/029424, filed Apr. 25, 2018, which claims the benefit of priority under 35 U.S.C. §119(e) to U.S. Provisional Application No: 62/489,913, filed Apr. 25, 2017, which is incorporated herein by reference in its entirety.

### INCORPORATION BY REFERENCE OF SEQUENCE LISTING

The contents of the text file named “52095-551N01US-SL.txt,” which was created on May 28, 2020 and is 19.3 KB in size, are hereby incorporated by reference in their entirety.

### FIELD OF THE INVENTION

The present invention relates generally to universal immunotherapy compositions useful for targeted treatment of cancers and other immune disorders.

### BACKGROUND OF THE INVENTION

Clinical trials have demonstrated that cancer immunotherapies can induce durable responses in patients with advanced cancers. One of the most successful cancer immunotherapies is the use of chimeric antigen receptor (CAR) T cells to treat B cell-derived leukemias and lymphomas. The chimera used against these cancers is a single chain antibody specific for CD19 fused to CD28 (a T cell co-stimulatory protein) and then fused to CD3zeta (T cell receptor (TCR) signaling protein). T cells expressing this construct receive primary and secondary signals and generate robust immune responses against all cells expressing CD19—including normal B cells. To date, CAR T cell therapies have demonstrated only modest success against solid tumors in part because it is very difficult to identify antigens that are uniquely expressed on tumors but not on untransformed cells. The current invention solves this problem.

### SUMMARY OF THE INVENTION

One of the biggest impediments to cancer immunotherapy is identifying antigens uniquely expressed on tumor cells but not expressed on normal, healthy cells. The present invention circumvents this challenge by leveraging several technological innovations to create an effective and universal CAR-T-based cell therapy system with significantly diminished off-target tissue effects.

The present invention utilizes in its various aspects, bifunctional compounds or complexes that contain at least two domains. One domain, referred to herein as a targeting moiety, binds an antigen on the surface of a tumor cell. The other domain, referred to herein as a pro-antigen, is designed to be inert to normal (non-diseased) cells and tissues, and to become activated (or “unmasked” or “uncaged”) only upon exposure to light of an appropriate wavelength. The present invention also utilizes chimeric antigen receptor (CAR) T cells. These T cells are equipped with mechanisms that kill cells to which they bind. The CAR-T cells of the present invention are designed to specifically bind the pro-antigen, but only in its unmasked or activated form. The introduction of light in and around a tumor causes activation of the pro-antigen. As a consequence, the CAR-T cells selectively target and eliminate diseased cells.

Accordingly, a first aspect of the present invention is directed to a bifunctional compound including a pro-antigen covalently linked to a targeting moiety. The pro-antigen, which constitutes one functional modality of the present compounds, is a small molecule having one or more photocleavable protecting groups. The targeting moiety, which constitutes a second functional modality of the present compounds, specifically binds a tumor associated antigen.

In some embodiments, the small molecule is a fluorescent molecule such as a fluorescein, an anthracene, an alexa fluor, a rhodamine, a rhodol, an acridine or a xanthene.

In some embodiments, the photocleavable protecting group is an ortho-nitrobenzyl group, a phenacyl ester group, an 8-quinolinyl benzenesulfonate group, a dicoumarin group, a 6-bromo-7-alkoxycoumarin-4-ylmethoxycarbonyl group, a bimane group or a bis-arylhydrazone group. In certain embodiments, the photocleavable protecting group is an ortho-nitrobenzyl group. In certain embodiments, the ortho-nitrobenzyl group protecting group is

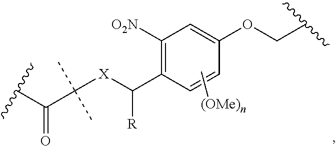

wherein X is NH or O, R is C1-4 alkyl or H and n is 0-3. In certain embodiments, the ortho-nitrobenzyl group protecting group is

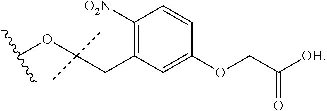

In some embodiments, the bifunctional compound has a formula (A) or (A′):

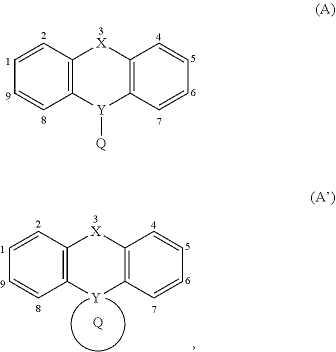

wherein X is C or O, Y is C or N, the photocleavable protecting group is present at one or more of positions 1-9 and Q represents one or more optionally substituted rings or a photocleavable protecting group. The optionally substituted rings are 4-7 carbocyclic or heterocyclic rings or a fused ring system, which may be saturated or non-saturated, and wherein heteroatoms may be selected from N, O and S.

In some embodiments, the bifunctional compound has a formula (I):

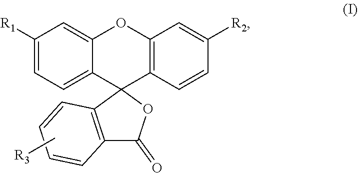

wherein R₁ is O, OH or a photocleavable protecting group,
R₂ is O, OH or a photocleavable protecting group; and
R₃ is

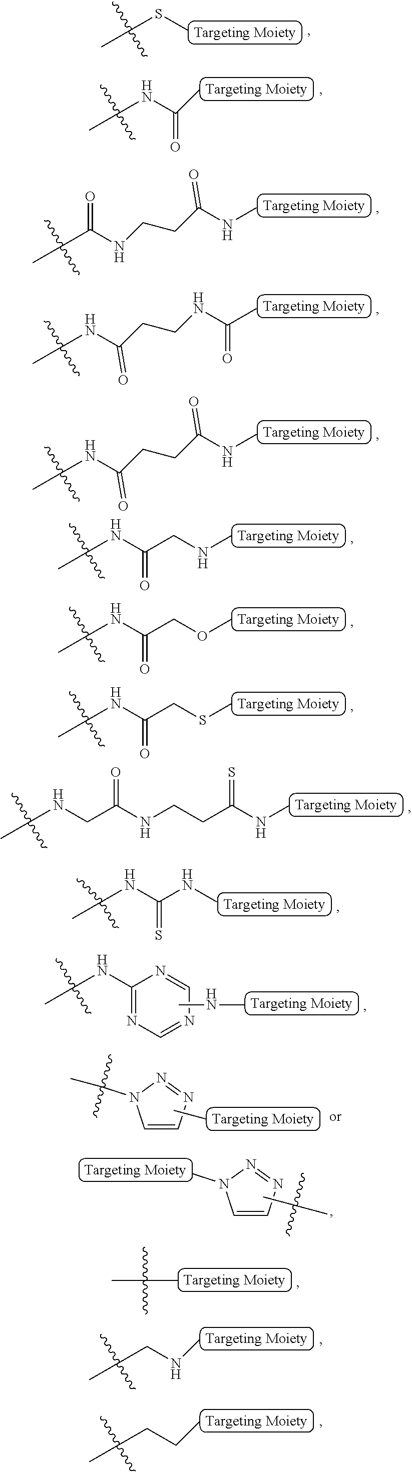

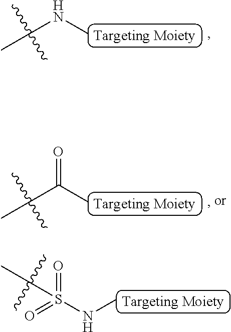

or stereoisomers thereof.

In some embodiments, the bifunctional compound has a formula (Ia):

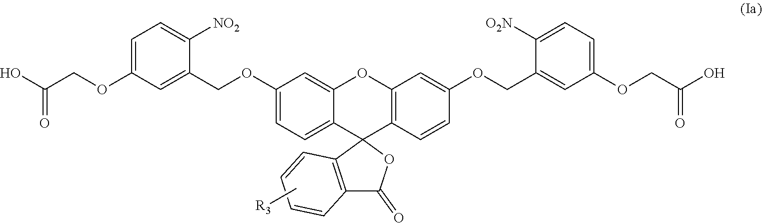

or stereoisomers thereof.

In some embodiments, the bifunctional compound has a formula (Ib):

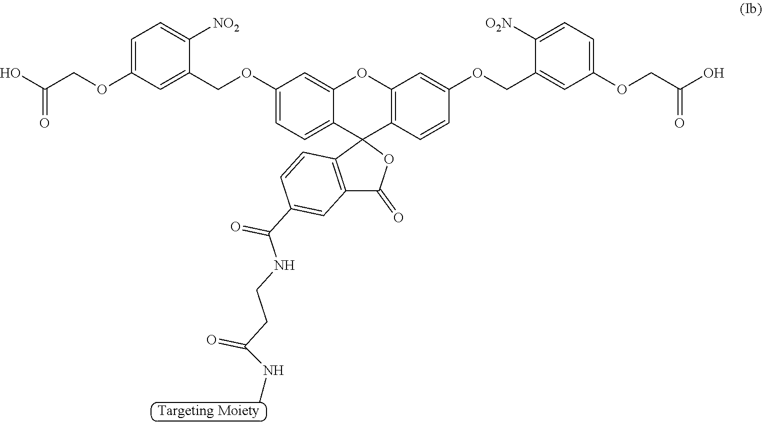 [SMILES: CNC(=O)CCNC(=O)c1ccc2c(c1)C(=O)OC21c2ccc(OCc3cc(OCC(=O)O)ccc3[N+](=O)[O-])cc2Oc2cc(OCc3cc(OCC(=O)O)ccc3[N+](=O)[O-])ccc21]

or stereoisomers thereof.

In some embodiments the bifunctional compound has a formula (II):

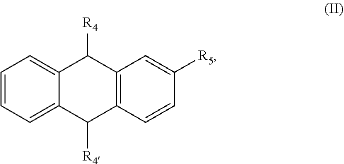

wherein each of R₄ and R₄′ is independently O or a photocleavable protecting group; and
Rs is

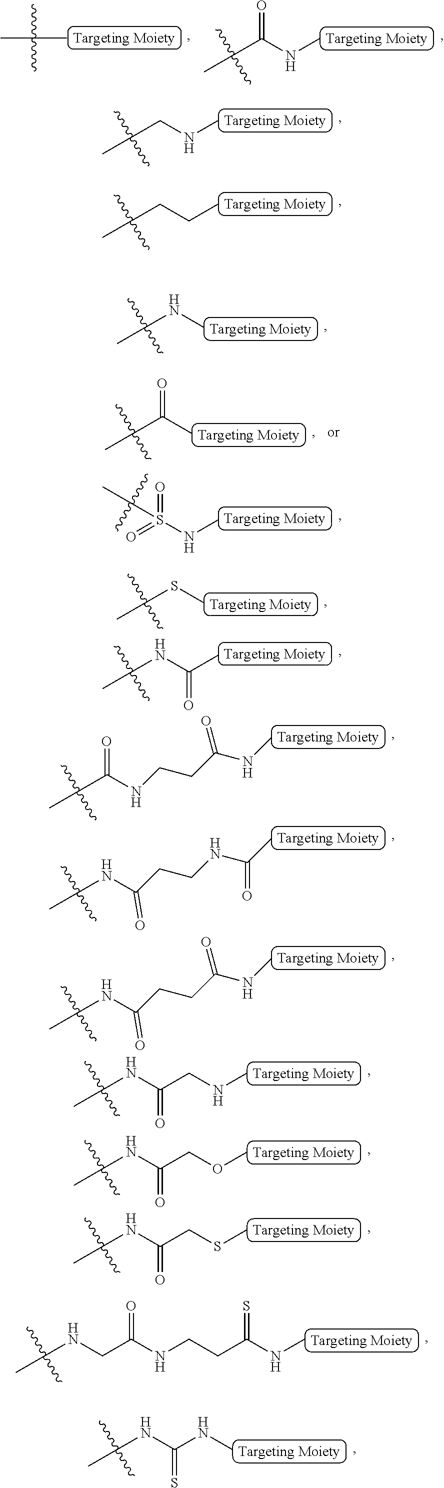

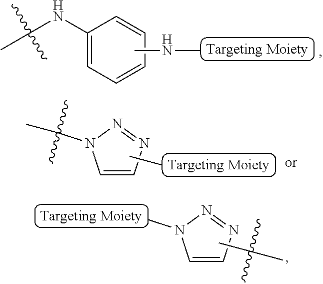

or stereoisomers thereof.

In some embodiments, the bifunctional compound has a formula (IIa):

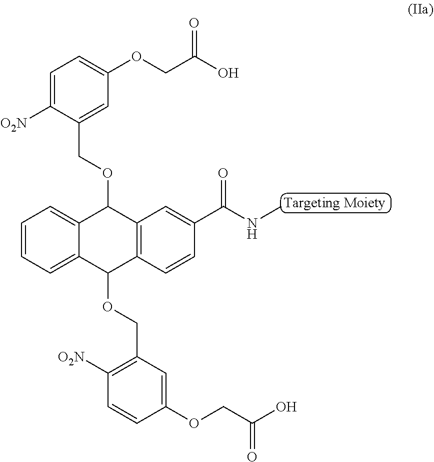 [SMILES: CNC(=O)c1ccc2c(c1)C(OCc1cc(OCC(=O)O)ccc1[N+](=O)[O-])c1ccccc1C2OCc1cc(OCC(=O)O)ccc1[N+](=O)[O-]]

or stereoisomers thereof.

In some embodiments, the targeting moiety is an antibody, an antibody fragment, a ligand, an aptamer or a nanobody.

In some embodiments, the targeting moiety specifically binds a tumor associated antigen on cancer cells. Representative examples of tumor associated antigens include platelet derived growth factor receptor alpha (PDGFRa), activin a receptor type 1 (ACVR1) human epidermal growth factor receptor 2 (HER2), prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, an abnormal p53 protein, mesothelin, EGFRvIII, EGFR1, diganglioside GD2, interleukin 13 receptor a (IL13Ra), fibroblast activation protein (FAP), and L1 cell adhesion molecule (L1CAM).

In some embodiments, the targeting moiety specifically binds a tumor associated antigen expressed or overexpressed on a light-accessible tumor. Representative examples of light-accessible tumors include those located in tissues of skin, cervix, bladder, prostate, bile duct, pancreas, stomach, brain, mouth, larynx, vagina, vulva or nasal passages.

In some embodiments, the targeting moiety is trastuzumab, cetuximab, panitumamab, zalutumumab, nimotuzumab, matuzumab, gefitinib, erlotinib, lapatinib, pertuzumab, tositumomab, rituximab, ibritumomab tiuxetan, daclizumab, CEA-scan, colo101, OC125 monoclonal antibody, Ab75705, an anti-AFP antibody or fragment thereof, humanized B3, B72.3, bevacizumab, an anti-CD99 antibody or fragment thereof, an anti-HER2 antibody or fragment thereof or an anti-EGFR antibody or fragment thereof.

In another aspect, pharmaceutical compositions of a therapeutically effective amount of the compound and a pharmaceutically acceptable carrier are also provided.

In another aspect, methods of making the compounds of the present application are also provided.

In another aspect, systems of the compounds and CAR-T cells are also provided.

In another aspect, kits containing one or more of the compounds are also provided. In some embodiments, the kits contain one or more of the compounds and reagents for producing autologous CAR-T cells that specifically recognize an unmasked compound of the invention. In some embodiments, the kits contain one or more of the compounds and allogeneic CAR-T cells that specifically recognize an unmasked compound of the invention.

In further aspects, methods of treating cancer are provided, which include administering to a subject in need thereof, a therapeutically effective amount of a compound of the invention, light at a wavelength appropriate to cleave the protecting group and a therapeutically effective number of chimeric antigen receptor T (CAR-T) cells, wherein the CAR-T cells include an extracellular ligand that specifically binds unmasked pro-antigen.

In some embodiments, the compound and light are administered to the subject prior to administration of the CAR-T cells. In other embodiments, the compound and light are administered to the subject after administration of the CAR-T cells. In yet other embodiments, the compound and light are administered to the subject concomitantly with administration of the CAR-T cells.

In some embodiments, the compound is administered at a dose of 0.01 mg/kg to 500 mg/kg body weight. In some embodiments, the CAR-T cells are administered at a dose of 10⁴ to 10⁹ cells per kg body weight. In some embodiments, the light is administered at a wavelength of 10 to 600 nm. In some embodiments, the CAR-T cells are administered parenterally. In some embodiments, the light is administered via a non-invasive procedure or a minimally-invasive procedure. In some embodiments, the light is administered during or after surgery. In some embodiments, the compound and the light are administered more than once and the CAR-T cells are administered once.

### BRIEF DESCRIPTION OF THE FIGURES

FIG. 1A shows the chemical structure of a fluorescein derivative masked with a photocleavable group conjugated to bovine serum albumin (BSA) and the unmasked product generated after exposure to light at a wavelength of 365 nm.

FIG. 1B is a representation of a chemical model showing binding of anti-fluorescein antibody (antibody 4M5.3) to unmasked fluorescein (FL). Anti-fluorescein antibody amino acid polar contacts are indicated by yellow dotted lines. The contacts with the amino acid side chains indicated in purple are presumed to be directly displaced when fluorescein is masked.

FIG. 1C is a graph showing specific activation of mouse anti-fluorescein CAR-T cells to the unmasked fluorescein derivative. BSA-conjugated masked fluorescein was coated onto a solid support, either in the absence of light activation (masked) or in the presence of light activation by exposure to 365 nm light (unmasked). Mouse anti-fluorescein CAR-T cells were co-incubated with the masked, unmasked, or BSA alone for ˜5 hrs. Mouse anti-fluorescein CAR-T cells upregulated the early activation marker CD69, only in the presence of the unmasked fluorescein (BSA-conjugated masked fluorescein that was exposed to 365nm light).

FIG. 2 is a drawing showing that anti-FL CAR-T cells will not recognize a caged FL molecule attached to an antibody that recognizes a tumor associated antigen.

FIG. 3 is a drawing depicting the unmasking of FL conjugated to a patient tumor targeting antibody upon exposure to light of the appropriate wavelength to cleave the protecting group.

FIG. 4 is a drawing depicting recognition of FL by anti-FL CAR-T cells after unmasking by exposure to light of the appropriate wavelength to cleave the protecting group.

FIG. 5A is a drawing depicting photo-cleavage of a protecting group consisting of a photocleavable linker and a polymer (light zig-zag line) to generate a small molecule antigen (star) attached to a tumor targeting moiety (dark zig-zag line).

FIG. 5B is a drawing depicting photo-cleavage of a protecting group consisting of a polymer (light zig-zag line) to generate a small molecule antigen (star plus photocleavable group) attached to a tumor targeting moiety (dark zig-zag line).

FIG. 6 is a graph showing specific in vitro cytotoxicity of human anti-fluorescein CAR-T cells to the unmasked fluorescein derivative. Human α-fluorescein CAR T cells were co-cultured with the human CD99+ Ewing Sarcoma cell line (A673) coated with anti-CD99 or a negative control antibody at an effector to target ratio of 20:1 for ˜4 hrs. A673 tumor cells were specifically killed when targeted with the anti-CD99 caged fluorescein unmasked by a pretreatment with 365nm light (uncaged), but not anti-CD99 caged fluorescein (caged). No statistically significant difference was observed between A673 cells targeted with caged anti-CD99 fluorescein and the negative control antibody (IgG isotype control FITC). Statistical significance was calculated using the Kruskal-Wallis one-way analysis of variance, plus preselected pairwise comparisons (*p<0.05). Specific killing was calculated using cells treated with their respective antibody in the absence of CAR T cells. Experiment was performed with 4 replicates.

FIG. 7 is a chemical schematic flow diagram of the chemical conjugation of a tumor targeting moiety containing primary amine groups to an NHS-ester fluorescein derivative.

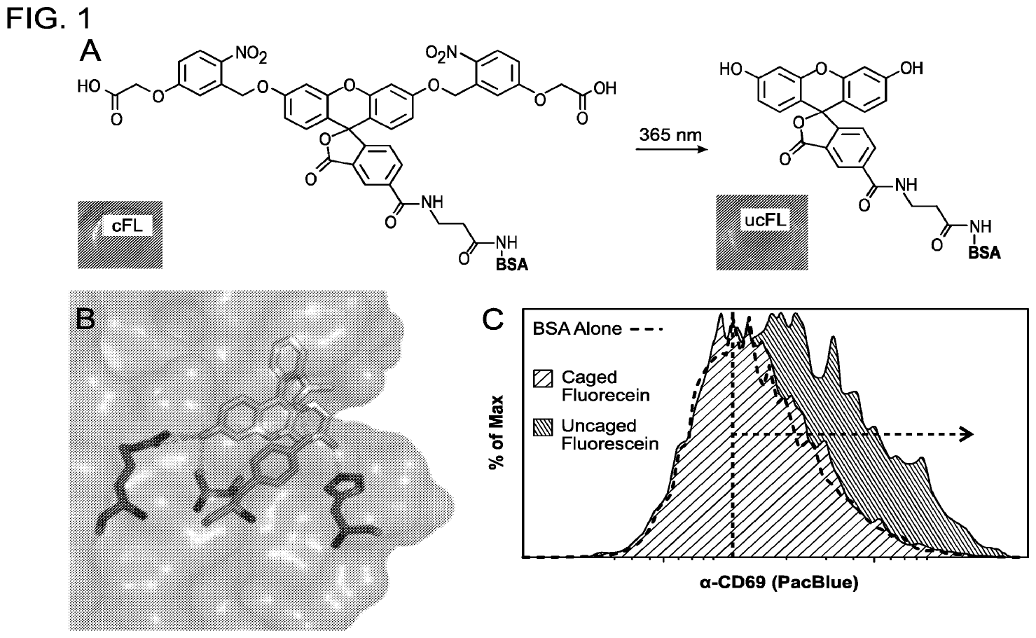

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In the case of conflict, the present specification, including definitions, will control. In addition, the materials, methods, and examples are illustrative only and not intended to be limiting.

The present invention relates to universal immunotherapy systems/kits, compositions and methods of treating cancer.

One of the biggest impediments to cancer immunotherapy is identifying antigens uniquely expressed on tumor tissue but not expressed on normal, healthy tissue. The present invention overcomes these challenges by leveraging several technological innovations to create an effective CAR T cell therapy system. Specifically, the present invention provides reagents that allow detection of any antigen on a tumor and generation of immune reactivity against that tumor without off-target tissue effects.

Briefly, in various aspects the current invention is composed of three parts (1) a tumor-targeting binding molecule (i.e., a targeting moiety), (2) a masked small molecule (i.e., a pro-antigen), and (3) a CAR-T cell specific for the small molecule. The mask is sensitive to photons that results in “unmasking” of the small molecule. Preferably the mask is photosensitive. For example, the mask is sensitive to UV light.

The current invention is an improvement over the invention disclosed in PCT/US2017/018216, the contents of which are incorporated by reference in its entirety. Specifically, unlike the previous disclosure in which the tumor itself programmed access (i.e., unmasking) to the small molecule, the present invention allows the physician to program access to the small molecule by directing the introduction of light of an appropriate wavelength to a tumor.

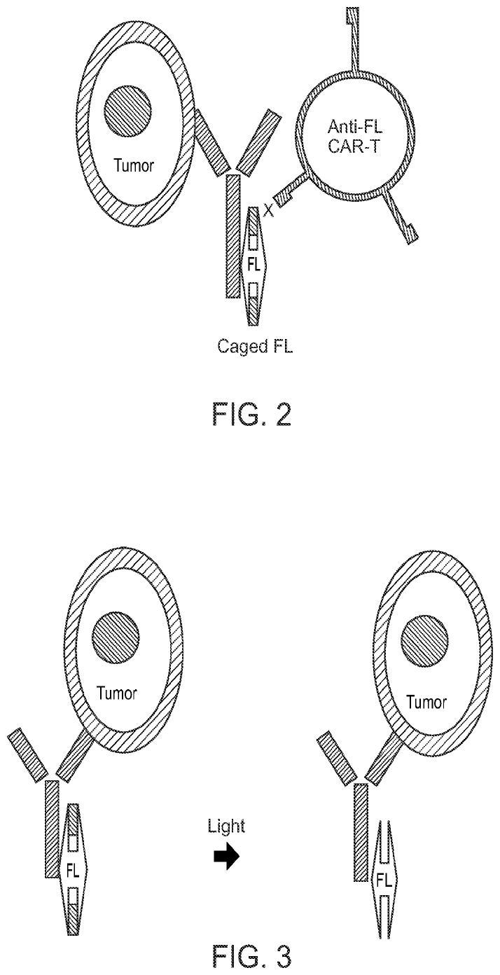

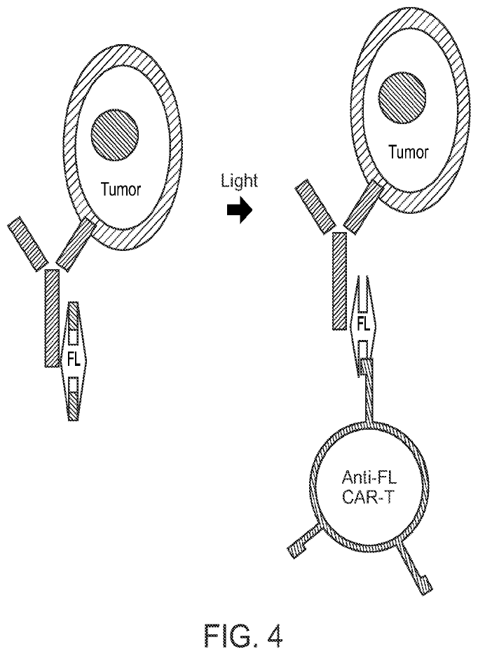

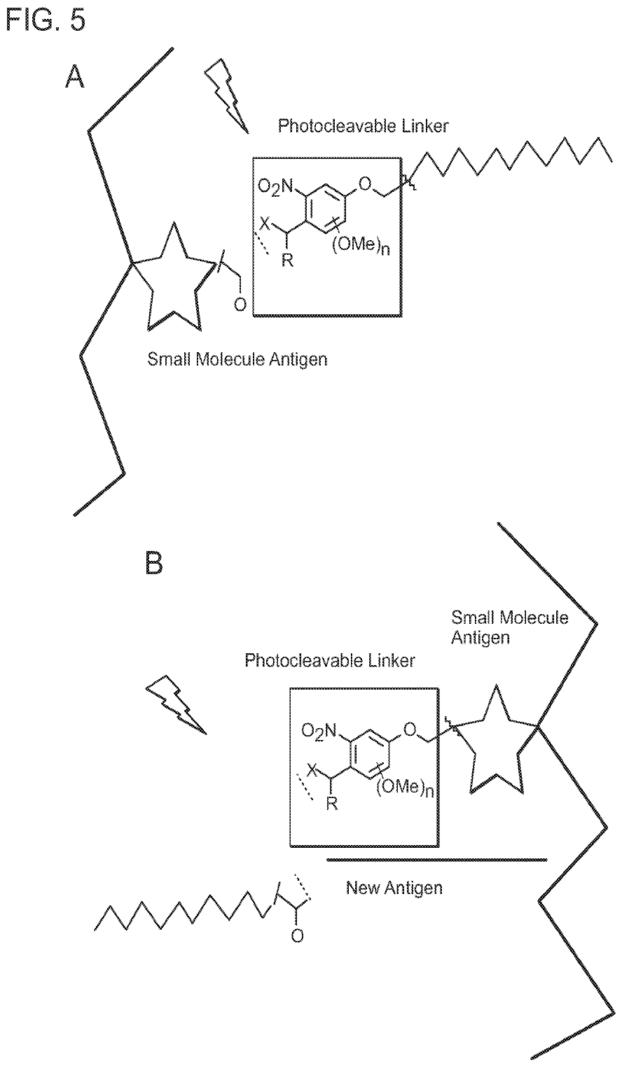

The overarching strategy is schematically presented in FIGS. 2-4, a series of binary events determines whether CAR T cells will be activated by the synthetic small molecule. These Binary Activated T cells using Chimeric Antigen Receptors (BAT-CARs) should be completely inert in the presence of the masked small molecule and activated only at sites where the small molecule is unmasked. The systems and compositions of the invention can be tailored to direct T cell responses against any solid tumor in a patient-specific fashion.

Another aspect of this invention is that any chimeric cellular receptor can be engineered to be stimulated by administration of a small molecule. That is, fusion of a single-chain antibody with any cellular receptor, can produce novel chimeric receptors. Thus, administration of a small molecule recognized by the single chain antibody can stimulate downstream effects in the target cell characteristic of stimulating the receptor with its natural ligand. In the current invention, T cell receptor signaling can be enabled by administration of a small molecule. That is, administration of a small molecule recognized by the single-chain antibody fused to T cell signaling molecules (for example but not uniquely CD28 and CD3 zeta) leads to hallmark changes in T cells representative to T cell receptor signaling. By making chimeras of a small molecule binding single chain antibody with any cellular receptor, specific biological outcomes can be induced by administration of a small molecule recognized by the single chain antibody.

The reagents according to the invention generate a T cell-directed immune response specifically within a tumor without prior information on neoantigens. Tumors are targeted with a targeting moiety for an antigen (e.g. a tumor associated antigen; TAA) that is enriched but not necessarily unique to tumors. This targeting moiety will be coupled to a small molecule. The small molecule serves as the target for a universal CAR T cell engineered with an extracellular binding domain that is specific for the small molecule. This universal CAR T cell referred to herein as a “Binary Activated T cells using Chimeric Antigen Receptors (BAT-CARs) is completely inert in the absence of the small molecule. Systemic treatment of the patient with the masked small molecule conjugated targeting moiety will deliver the small molecule to the tumor, creating a unique target for the BAT-CARS. To prevent off target activation of the BAT-CAR T cell the small molecule is masked or caged with a photosensitive group. While intact, the cage prevents the small molecule from binding and activating the BAT-CAR T cell. A physician controls activation by administering light, of an appropriate wavelength to unmask the small molecule, at sites containing cancerous tissue.

Targeting Moiety

Targeting moieties according to the invention have binding specificity for tumor associated antigens (TAAs). A targeting moiety may also be referred to herein as a “binding molecule” or a “tumor targeting unit”. The term “tumor associated antigen”, which may also be referred to herein as a “tumor antigen” or a “cancer associated antigen”, refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell.

The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules that are able to bind to or recognize a binding partner (or a limited number of binding partners) to a substantially higher degree than to other, similar biological entities.

Representative examples of targeting moieties include antibody molecules and functional (i.e., antigen-binding) fragments thereof, receptor ligands, peptides, haptens, aptamers, affimers, T-cell receptor tetramers and other targeting molecules known to those skilled in the art. For example, the targeting moiety may include a nucleic acid, polypeptide, glycoprotein, carbohydrate, or lipid.

In certain embodiments, the targeting moiety is an antibody or antibody fragment. For example, an antibody includes monoclonal antibodies, polyclonal antibodies, Fv, Fab, Fab′ and F(ab′)₂ immunoglobulin fragments, synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv, single domains from camelids or engineered human equivalents. Antibodies are made either by conventional immunization (e.g., polyclonal sera and hybridomas), or as recombinant fragments, usually expressed in E. coli, after selection from phage display or ribosome display libraries. Alternatively, ‘combibodies’ comprising non-covalent associations of VH and VL domains, can be produced in a matrix format created from combinations of diabody-producing bacterial clones. The term “antibody” also includes any protein having a binding domain which is homologous or largely homologous to an immunoglobulin binding domain. Such proteins may be derived from natural sources, or partly or wholly synthetically produced.

In certain embodiments, the targeting moiety is an affimer. An affimer is a small, highly stable protein engineered to display peptide loops which provide a high affinity binding surface for a specific target protein. It is a protein of low molecular weight, 12-14 kDa, derived from the cysteine protease inhibitor family of cystatins. Affimer proteins are composed of a scaffold, which is a stable protein based on the cystatin protein fold. They display two peptide loops and an N-terminal sequence that can be randomized to bind different target proteins with high affinity and specificity similar to antibodies. Stabilization of the peptide upon the protein scaffold constrains the possible conformations which the peptide may take, thus increasing the binding affinity and specificity compared to libraries of free peptides.

In certain embodiments, the targeting moiety is a nucleic acid binding molecule (e.g. an aptamer) that binds to a cell type specific marker. In general, an aptamer is an oligonucleotide (e.g., DNA, RNA, or an analog or derivative thereof) that binds to a particular target, such as a polypeptide. Aptamers are short synthetic single-stranded oligonucleotides that specifically bind to various molecular targets such as small molecules, proteins, nucleic acids, and even cells and tissues. These small nucleic acid molecules can form secondary and tertiary structures capable of specifically binding proteins or other cellular targets, and are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment) (Ellington et al. Nature. 1990; 346(6287):818-822; Tuerk et al., Science. 1990; 249(4968):505-510; Ni et al., Curr Med Chem. 2011; 18(27):4206-14; which are incorporated by reference herein in their entireties). Methods of generating an apatmer for any given target are well known in the art.

In some embodiments, a targeting moiety may be a naturally occurring or synthetic ligand for a cell surface receptor.

In some embodiments, the targeting moiety is a carbohydrate. Carbohydrates may be natural or synthetic. A carbohydrate may be a derivatized natural carbohydrate. In some embodiments, the carbohydrate comprises monosaccharide or disaccharide, including but not limited to, glucose, fructose, galactose, ribose, lactose, sucrose, maltose, trehalose, cellbiose, mannose, xylose, arabinose, glucoronic acid, galactoronic acid, mannuronic acid, glucosamine, galatosamine, or neuramic acid. In some embodiments, the carbohydrate is a polysaccharide, such as, but not limited to, pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate is a sugar alcohol, such as, but not limited to mannitol, sorbitol, xylitol, erythritol, maltitol, or lactitol.

Representative examples of targeting moieties are set forth in Table 1:

TABLE 1

| Tumors | Targeting Moieties |
|---|---|
| Adenocarcinoma (eg., | Cetuximab, panitumamab, |
| colorectal cancer, head | zalutumumab, nimotuzumab, |
| and neck cancer) | matuzumab, gefitinib, |
| | erlotinib, lapatinib |
| Breast cancer, ovarian cancer, | Trastuzumab (Herceptin ®), |
| stomach cancer, uterine | pertuzumab |
| cancer | |
| Non-Hodgkin lymphoma | Tositumomab (Bexxar ®), |
| | Rituximab (Rituxan, |
| | Mabthera), Ibritumomab |
| | tiuxetan (Zevalin) |
| T-cell lymphoma | Daclizumab (Zenapax) |
| Colorectal cancer, some gastric | CEA-scan (Fab fragment, |
| cancers, biliary cancer | approved by FDA), |
| | colo101 |
| Ovarian cancer, mesothelioma, | OC125 monoclonal antibody |
| breast cancer | |
| Hepatocellular carcinoma | Ab75705 (available from |
| | Abcam) and other |
| | commercially available |
| | AFP antibodies |
| Colorectal cancer, biliary cancer | B3 (humanized) |
| Adenocarcinomas including | B72.3 (FDA-approved |
| colorectal, pancreatic, gastric, | monoclonal antibody) |
| ovarian, endometrial, | |
| mammary, and non-small | |
| cell lung cancer | |
| Colorectal cancer | Bevacizumab (Avastin ®) |
| Ewing sarcoma | Anti-CD99 antibody or |
| | fragment thereof |
| Breast cancer | Anti-HER2 or anti-EGFR |
| | antibody or fragment |
| | thereof |

Targeting moieties according to the invention have binding specificity for tumor associated antigens. By tumor associated antigen it is meant any cell surface molecule or combination of molecules on a tumor cell. Preferably, the tumor antigen distinguishes tumor cells from normal cells. Tumor antigens can distinguish tumor cells from normal cell by being uniquely expressed on tumor cells or over-represented in tumor cells compared to normal cells. A tumor antigen is a polypeptide, a peptide (e.g. MHC peptide), a lipid, or a carbohydrate.

In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, the tumor associated antigen is a MHC presented peptide. Normally, peptides derived from endogenous proteins fill the pockets of Major histocompatibility complex (MHC) class I molecules, and are recognized by T cell receptors (TCRs) on CD8+lymphocytes. The MHC class I complexes are constitutively expressed by all nucleated cells. In cancer, virus-specific and/or tumor-specific peptide/MHC complexes represent a unique class of cell surface targets for immunotherapy.

Targeting moieties that bind TAAs are known in the art. In certain embodiments, the targeting moiety is directed to a TAA expressed by tumors that are accessible to administered light (“light-accessible tumors”). Representative examples of light-accessible tumors include those found in the tissues of skin, cervix, bladder, prostate, bile duct, pancreas, stomach, brain, mouth, larynx, vagina, vulva and nasal passages. In some embodiments, light may be delivered to some tumors via interstitial therapy, which involves using imaging tests (such as CT scans) to guide fiber optics directly into tumors using needles or other minimally invasive means. Light delivered via interstitial therapy can be used to treat tumors found in the breast, ovaries, head and neck, prostate, liver and lungs. In other embodiments, light can be delivered to tumor tissue during or after surgery to target any cancerous tissues that may be remaining after tumor excision.

Representative examples of tumor associated antigens (TAAs) to which the targeting moiety may be directed include platelet derived growth factor receptor alpha (PDGFRa), activin a receptor type 1 (ACVR1) human epidermal growth factor receptor 2 (HER2), prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, an abnormal p53 protein, mesothelin, EGFRvIII, EGFR1, diganglioside GD2, interleukin 13 receptor a (IL13Ra), fibroblast activation protein (FAP), and L1 cell adhesion molecule (L1CAM).

Pro-Antigen

The pro-antigens of the present invention are small molecules that contain a photocleavable protection group. The pro-antigen, which is also referred to herein as a “masked tag”, “caged tag”, “masked recognition domain” or “caged recognition domain”, is inert in the sense that it does not bind and activate CAR-T cells unless it becomes unmasked or uncaged upon exposure to an appropriate wavelength of light.

Tag/Recognition Domain

The “tag” or “recognition domain” serves as the target for a universal CAR T cell. The recognition domain is also referred to herein as an “antigen small molecule” or “small molecule”. The recognition domain is linked to the targeting moiety in such a manner as not to interfere with the ability of the targeting moiety to bind to its ligand, eg. a TAA. The recognition domain is one or more (i.e., plurality) of small molecules. The small molecule is synthetic or naturally-occurring. The small molecule is biologically active or inactive. As used herein, the phrase “biologically active” refers to a characteristic of any substance that has activity in a biological system and/or organism. For instance, a substance that, when administered to an organism, has a biological effect on that organism, is considered to be biologically active.

In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol.

Representative examples of small molecules that can be linked to the targeting moieties include a fluorescein, an anthracene, a rhodamine, a rhodol, an alexa fluor, an acridine, a xanthene, a pyrazine, an amphetamine, a benzodiazepine, a benzoylecgonine, a buprenorphine, an opioid, a cannabinoid, a phencyclidine, a tricyclic antidepressant, dextromethorphan, fentanyl, meprobamate, methadone, methamphetamine, oxycodone, THC, tramadol, zolpidem, ketamine, LSD, MDMA, methaqualone, propoxyphene or norketimine. Other exemplary small molecules for use in the present invention include those listed at http://www.randoxtoxicology.com/products/biochip-array and http://www.randoxtoxicology.com/products/biochip-array/doa-1.

Representative examples of fluorescein derivatives that can be linked to the targeting moieties include 5-carboxyfluorescein, 6-carboxyfluorescein, 5-(iodoacetamido)fluorescein, 5-([4,6-dichlorotriazin-2-yl]amino)fluorescein hydrochloride, 5-(bromomethyl)fluorescein, fluorescein 5(6)-isothiocyanate, and fluorescein 5-carbamoylmethylthiopropanoic acid.

Representative examples of anthracene derivatives that can be linked to the targeting moieties include anthraquinone, anthraquinone-2-carboxylate, 2-aminoanthraquinone, 2-iodoanthraquinone, 2-chloroanthraquinone, 2-bromoanthraquinone, 2-ethynylanthraquinone, 2-cyanoanthraquinone, anthraquinone-2-sulfonate, anthraquinone-2-carbonyl chloride and 2-hydroxyanthraquinone.

The compounds of the present invention may be prepared by conjugating the targeting moiety to the pro-antigen using techniques such as chemical coupling and chemical cross-linkers. In some embodiments, the pro-antigen may be conjugated to the targeting moiety via a linker. Representative examples of aliphatic linkers include glycine, aminoheptanoic acid, aminohexanoic acid, aminopentanoic acid and aminotetranoic acid. Representative examples of polar linkers include polyethylene glycol with 2, 4 or 6 repeating units.

Protection Domain

The tag or recognition domain is linked to a protection domain. The protection domain is also referred to herein as the “mask” or “cage”. The protection domain serves to mask the recognition domain to prevent the recognition domain from binding and activating the CAR-T cells. The protection domain is in whole or in part photosensitive, i.e. photocleavable. As used herein, the terms “photosensitive” and “photocleavable” are interchangeable. In some embodiments, the protection domain is composed of one or more photocleavable groups. When the photocleavable groups are exposed to light, the pro-antigen is unmasked thereby generating a tag or recognition domain.

In some aspects, the protection domain is composed of a photocleavable group and a masking polymer.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d′Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference).

Representative examples of polymers include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Representative examples of non-biodegradable polymers include ethylene vinyl acetate, poly(meth)acrylic acid, polyamides, copolymers and mixtures thereof.

Representative examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycoloic acid and blends thereof.

PVP is a non-ionogenic, hydrophilic polymer having a mean molecular weight ranging from approximately 10,000 to 700,000 and the chemical formula (C₆H₉NO)ₙ. PVP is also known as poly[1-(2-oxo-1-pyrrolidinyl)ethylene], Povidone™, Polyvidone™, RP 143™, Kollidon™, Peregal ST™, Periston™, Plasdone™, Plasmosan™, Protagent™ Subtosan™, and Vinisil™. PVP is non-toxic, highly hygroscopic and readily dissolves in water or organic solvents.

Polyethylene glycol (PEG), also known as poly(oxyethylene)glycol, is a condensation polymer of ethylene oxide and water having the general chemical formula HO(CH₂CH₂O)ₙH.

Polyvinyl alcohol (PVA) is a polymer prepared from polyvinyl acetates by replacement of the acetate groups with hydroxyl groups and has the formula (CH₂CHOH)ₙ. Most polyvinyl alcohols are soluble in water. PEG, PVA and PVP are commercially available from chemical suppliers such as the Sigma Chemical Company (St. Louis, Mo.).

In certain embodiments the polymer may comprise poly(lactic-co-glycolic acid) (PLGA).

Representative examples of photocleavable groups include ortho-nitrobenzyl based groups, phenacyl ester based groups, 8-quinolinyl benzenesulfonate group, dicoumarin group, 6-bromo-7-alkoxycoumarin-4-ylmethoxycarbonyl group, bimane based groups, and bis-arylhydrazone based groups. General structures and cleavage conditions are as follows:

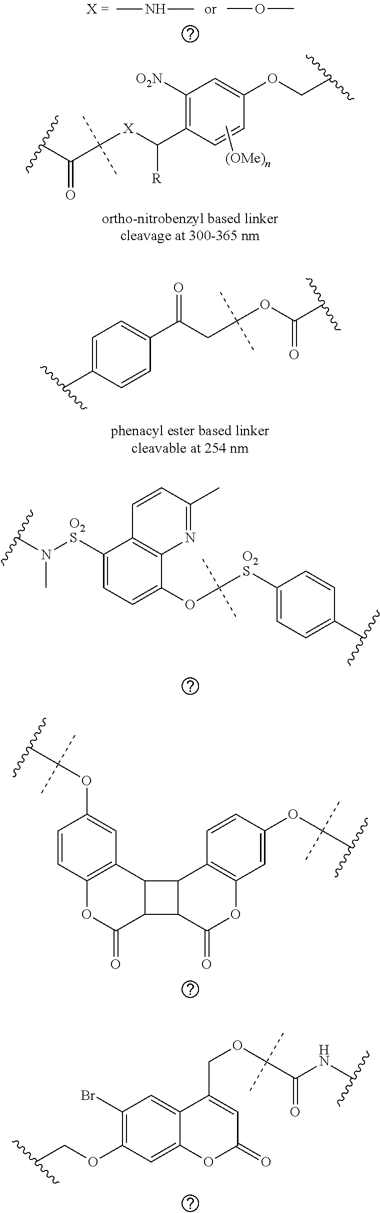

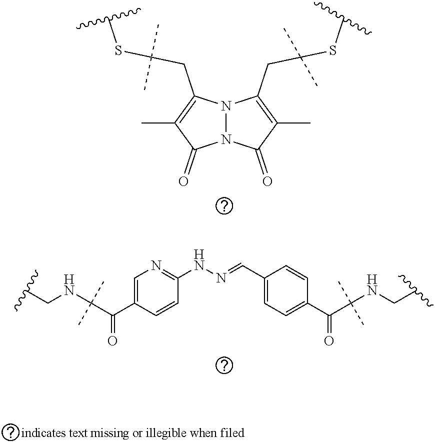

In certain embodiments, the photocleavable protecting group is

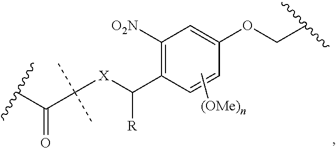

wherein X is NH or O, R is C1-4 alkyl or H and n is 0-3. In some embodiments, the ortho-nitrobenzyl based group is

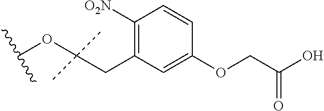

cleavage at 300-365 nm.

Dashed lines indicate sites of cleavage.

Bifunctional Compounds

In some embodiments, the bifunctional compound has a formula (A) or (A′):

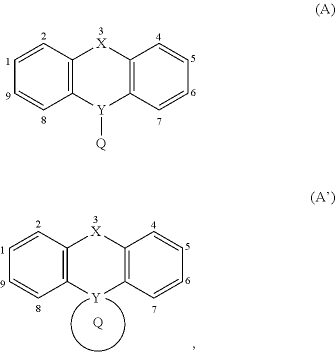

wherein X is C or O, Y is C or N, the photocleavable protecting group is present at one or more of positions 1-9 and Q represents one or more optionally substituted rings or a photocleavable protecting group. The optionally substituted rings are 4-7 carbocyclic or heterocyclic rings or a fused ring system, which may be saturated or non-saturated, and wherein heteroatoms may be selected from N, O and S.

In some embodiments, the bifunctional compound has a formula (I):

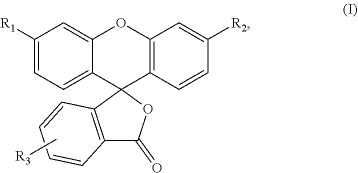

wherein R₁ is O, OH or a photocleavable protecting group,
R₂ is O, OH or a photocleavable protecting group; and
R3 is

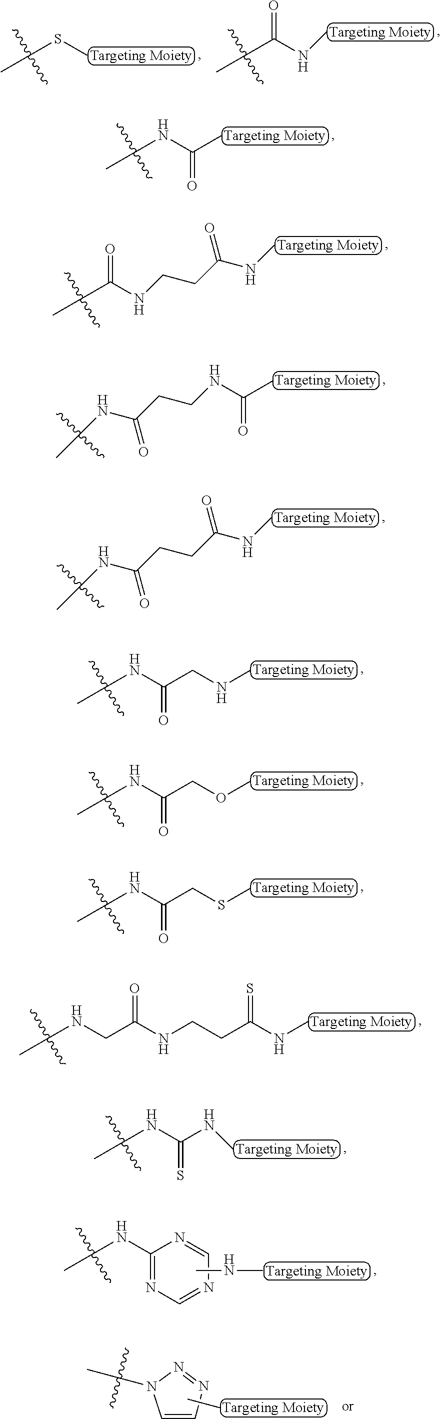

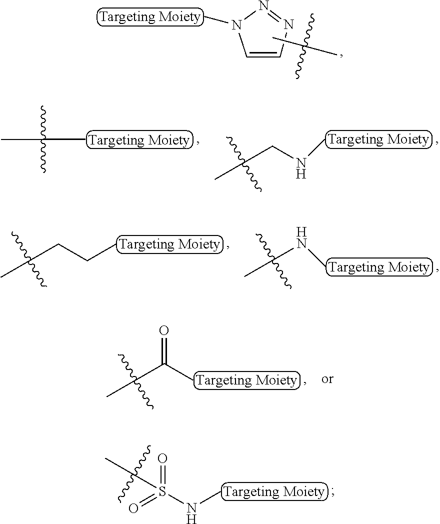

or stereoisomers thereof.

In some embodiments, the bifunctional compound has a formula (Ia):

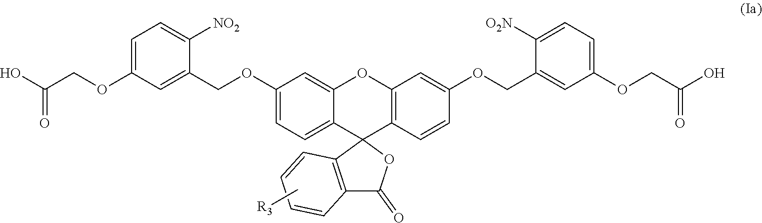

or stereoisomers thereof

In some embodiments, the bifunctional compound has a formula (Ib):

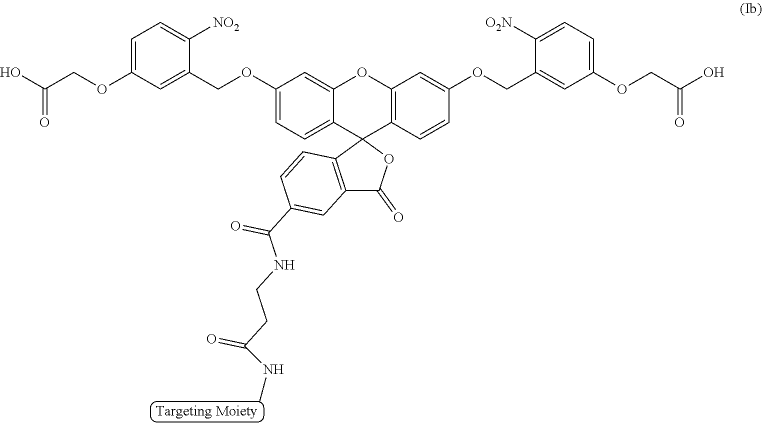 [SMILES: CNC(=O)CCNC(=O)c1ccc2c(c1)C(=O)OC21c2ccc(OCc3cc(OCC(=O)O)ccc3[N+](=O)[O-])cc2Oc2cc(OCc3cc(OCC(=O)O)ccc3[N+](=O)[O-])ccc21]

or stereoisomers thereof

In some embodiments the bifunctional compound has a formula (II):

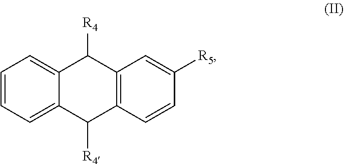

wherein each of R₄ and R₄′ is independently O or a photocleavable protecting group; and
R₅ is

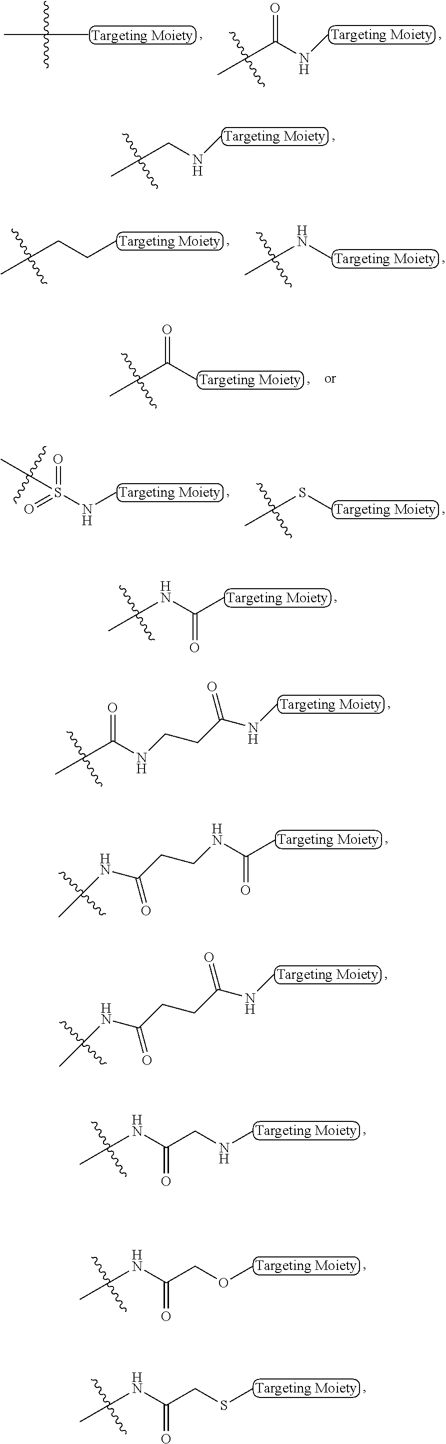

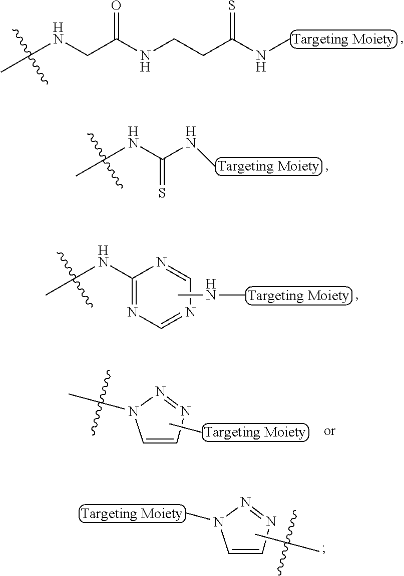

or stereoisomers thereof.

In some embodiments, the bifunctional compound has a formula (IIa):

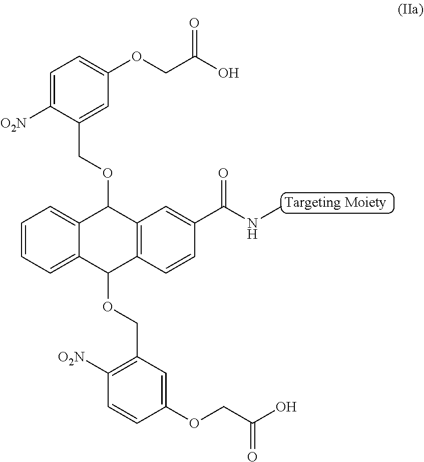 [SMILES: CNC(=O)c1ccc2c(c1)C(OCc1cc(OCC(=O)O)ccc1[N+](=O)[O-])c1ccccc1C2OCc1cc(OCC(=O)O)ccc1[N+](=O)[O-]]

or stereoisomers thereof.

Compounds of the present application may be in the form of a stereoisomer, which as used herein, embraces all isomers of individual compounds that differ only in the orientation of their atoms in space. The term stereoisomer includes mirror image isomers (enantiomers) of compounds, mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers).

Thus, the compounds of the present application may be in the form of individual isomers and substantially free of other isomers, and in the form of a mixture of various isomers, e.g., racemic mixtures of stereoisomers.

As used herein, “cyclic” refers to cycloalkyl, heterocycloalkyl, aryl or heteroaryl.

As used herein the term “cycloalkyl” alone or in combination with other term(s) means C3-C10 saturated cyclic hydrocarbon ring. A cycloalkyl may be a single ring, which typically contains from 3 to 7 carbon ring atoms. Examples of single-ring cycloalkyls include cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclic carbocyclyls.

The term “heterocycloalkyl” refers to a non-aromatic, saturated or partially saturated, monocyclic or polycyclic ring system of 3 to 15 members having at least one heteroatom or heterogroup selected from O, N, S, S(O), S(O)2, NH or C(O) with the remaining ring atoms being independently selected from the group consisting of carbon, oxygen, nitrogen and sulfur. Examples of “heterocycloalkyl” include azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, azepanyl, 2-azabicyclo[2.2.2]octanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups.

As used herein, the term “aryl” alone or in combination with other term(s) means a carbocyclic aromatic system containing one or two rings wherein such rings may be fused. The term “fused” means that the second ring is attached or formed by having two adjacent atoms in common with the first ring. The term “fused” is equivalent to the term “condensed”. Examples of aryl groups include phenyl, naphthyl, indanyl and the like. Unless otherwise specified, all aryl groups described herein may be substituted or unsubstituted.

The term “heteroaryl” refers to an aromatic heterocyclic ring system containing 5 to 20 ring atoms, suitably 5 to 10 ring atoms, which may be a single ring (monocyclic) or multiple rings (bicyclic, tricyclic or polycyclic) fused together or linked covalently. Preferably, “heteroaryl” is a 5- to 6-membered ring. The rings may contain from 1 to 4 heteroatoms selected from N, O and S, wherein the N or S atom is optionally oxidized, or the N atom is optionally quartemized. Any suitable ring position of the heteroaryl moiety may be covalently linked to the defined chemical structure.

Examples of heteroaryl include furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzisoxazolyl; benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, .alpha.-carboline, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and the like. Preferably “heteroaryl” refers to 5- to 6-membered ring selected from the group consisting of furanyl, thiophene, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl and pyridazinyl. More preferably, pyrazolyl, pyridyl, oxazolyl and furanyl. All heteroaryls are optionally substituted by one or more aforesaid groups.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including halo, cyano, alkyl, haloalkyl, alkoxy, haloalkoxy, alkenyl, alkynyl, aryl, heterocyclyl, amino, cyano, nitro, alkylamino, arylamino, alkylaminoalkyl, arylaminoalkyl, hydroxyl, hydroxyalkyl, cycloalkyl, aryl, heterocyclic and aliphatic. It is understood that the substituent may be further substituted.

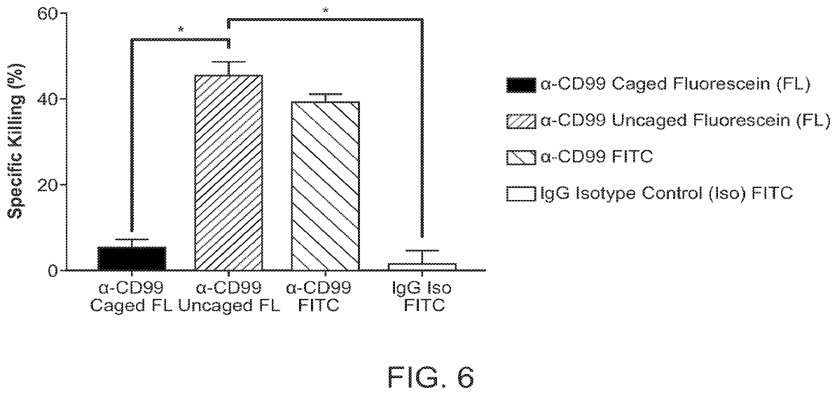

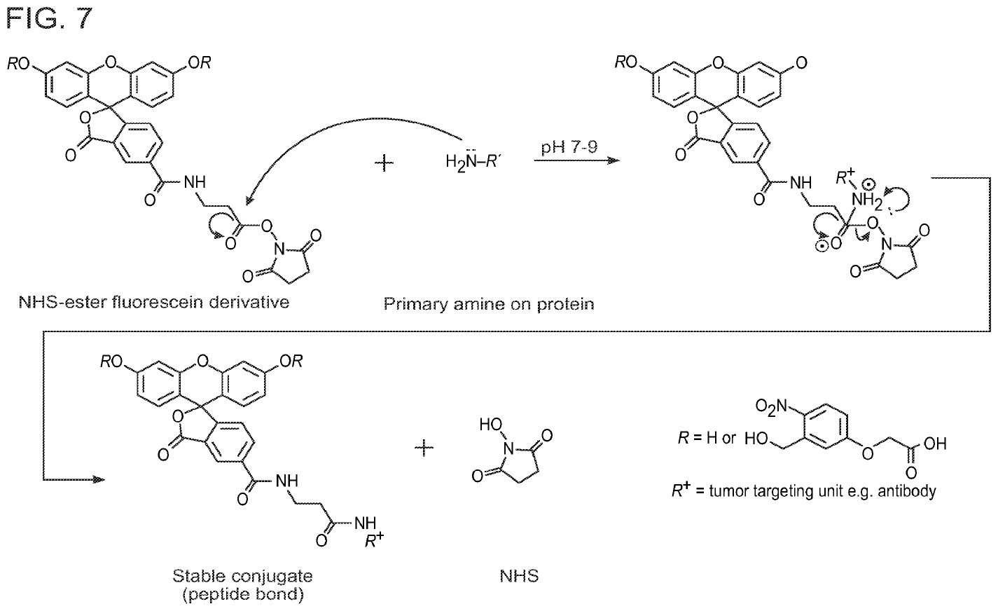

Bifunctional compounds of the present invention may be synthesized in accordance with methods known in the art. See, e.g., WO2010/008519. In general, the small molecules of the invention possess or can be derivatized to possess chemical groups that react with primary amines (e.g., the N-terminal amine or lysines of polypeptides) or with sulfhydryl groups (e.g., cysteines of polypeptides), of the polypeptides making up the targeting moieties of the bifunctional compounds. For example, see FIG. 6. The bifunctional compounds of the present invention may also be synthesized using click chemistry.

CARs

The effector cells used in the methods of the present invention may be autologous, syngeneic or allogeneic, with the selection dependent on the disease to be treated and the means available to do so. Suitable populations of effector cells that may be used in the methods include any immune cells with cytolytic activity, such as T cells. Exemplary sub-populations of T cells include those expressing CD3⁺ such as CD3⁺ CD8⁺ T cells, CD3⁺ CD4+ T cells, and NKT cells. Although in some embodiments the T cells are HLA-A2+ peripheral blood mononuclear cells (PBMC), they can be of any HLA background from PBMCs and utilized in an autologous, syngeneic or allogeneic system. T cells may also be isolated from any source, including from a tumor explant of the subject being treated or intra-tumoral T cells of the subject being treated. For the sake of convenience, the effector cells are hereinafter referred to as T cells, but it should be understood that any reference to T cells, unless otherwise indicated, is a reference to all effector cell types as defined herein.

The genetically engineered T cells used in the present invention allow for great flexibility. They have binding specificity for a particular unmasked pro-antigen (also referred to herein as a tag) that is conjugated to a targeting moiety (such as an antibody or functional fragment thereof) that binds to a tumor-associated antigen (TAA). Additional features of the CAR may include an activation domain that induces efficient target lysis upon T cell binding and activation, and the ability to substitute or replace the scFv portion of the CAR with one having specificity to any one of the unmasked pro-antigens or tags of the present invention. Since the unmasked pro-antigen serves as the target for a CAR T cell that is engineered with an extracellular binding domain that specifically binds the unmasked pro-antigen, and thus indirectly to the TAA, the CAR-T cells used in the invention may be referred to as universal CAR-T cells or Binary Activated T cells (BAT-CARs).

The BAT-CAR polypeptides typically include three domains. The first domain is an extracellular ligand or a tag-binding domain. This domain is typically present at the amino terminal end of the BAT-CAR polypeptide, and thus external to the T cell, which permits the tag-binding domain unfettered access to the tagged protein that is bound to the target cell. The tag-binding domain is typically an antibody or an antigen-binding fragment thereof. In some embodiments, the antibodies are human or humanized antibodies.

The tag-binding domain is designed to specifically bind the unmasked pro-antigen that is covalently linked to the targeting moiety that binds the target cells (e.g., cancer cells). For example, when the tag or unmasked pro-antigen is fluorescein or a fluorescein derivative, the tag-binding domain specifically binds fluorescein or a fluorescein derivative, examples of which are known in the art, e.g., 4M5.3 ScFv, disclosed in Midelfort, et al. J. Mol. Biol. 343:685-701 (2004).

The type of antibody, is not critical; it may also be polyclonal, monoclonal, chimeric or humanized. The antibodies may be obtained from any species of animal, e.g., a human, simian, mouse, rat, rabbit, guinea pig, horse, cow, sheep, goat, pig, dog or cat. Nor is there a limitation on the particular class of antibody that may be used, including IgGi, IgG₂, IgG₃, IgG₄, IgM, IgAi, IgA₂, IgD and IgE antibodies. Antibody fragments, which also may be used, include single-chain variable fragment (scFv), single chain antibodies, F(ab′)₂ fragments, Fab fragments, and fragments produced by an Fab expression library, provided that the antibody fragments retain the ability to bind the selected tag.

BAT-CARs of the present invention may be produced using commercially-available extracellular ligands, at least to the extent that the unmasked pro-antigens are known. Alternatively, antibodies and fragments thereof that specifically bind the unmasked pro-antigen can be prepared using standard techniques, e.g., continuous cell lines in culture for monoclonal antibody production. Representative techniques include the hybridoma technique originally described by Koehler and Milstein (Nature 256:495-497 (1975)), the human B-cell hybridoma technique (Kosbor et al., Immunol Today 4:72 (1983); Cote et al., Proc Natl. Acad. Sci 80:2026-2030 (1983)), and the EBV-hybridoma technique (Cole et al., Monoclonal Antibodies and Cancer Therapy, Alan R. Liss Inc, New York N.Y., pp 77-96 (1985)). Techniques developed for the production of “chimeric antibodies,” i.e., the splicing of mouse antibody genes to human antibody genes to obtain a molecule with appropriate antigen specificity and biological activity, can also be used (Morrison et al., Proc Natl. Acad. Sci 81:6851-6855 (1984); Neuberger et al., Nature 312:604-608(1984); Takeda et al., Nature 314:452-454(1985)). As known in the art, a humanized antibody or antibody fragment has one or more amino acid residues, typically from a variable domain of an antibody from a nonhuman source. Humanized antibodies or antibody fragments may contain one or more CDRs from nonhuman immunoglobulin molecules, and framework regions that are derived completely or mostly from human germline. Techniques for humanizing antibodies or antibody fragments are well known, and include CDR grafting, veneering or resurfacing, and chain shuffling. See, also, Jones et al., Nature 321:522-525 (1986); Riechmann et al., Nature, 332:323-327 (1988); Verhoeyen et al., Science, 239:1534-1536 (1988)).

In some embodiments, the tag-binding domain of the BAT-CAR-T is a single-chain variable fragment (scFv). A scFv includes the variable regions of the heavy (VH) and light chains (VL) of an antibody. A linker can be used to either connect the N-terminus of the VH with the C-terminus of the VL, or vice versa. ScFvs can be prepared according to methods known in the art (see, e.g., Bird et al., (1988) Science 242:423-426 (1988) and Huston et al., (1988) Proc. Natl. Acad. Sci. USA 85:5879-5883 (1988)). ScFv's can be produced by linking VH and VL regions together, typically by using flexible polypeptide linkers having from 1 to 50, e.g., 10 to 25 or 5 to 10 amino acid residues. In some embodiments, the linker sequence includes amino acids glycine and serine, and in some cases, sets of glycine and serine repeats such as (Gly₄Ser)ₙ, where n is an integer equal to or greater than 1. The length and amino acid composition of the linker may be varied e.g., to achieve optimal folding and interaction between the VH and VL to create a functional epitope. See, e.g., Hollinger et al. 1993 Proc Natl Acad. Sci. U.S.A. 90:6444-6448 (1993).

Other types of antibody fragments having specificity for unmasked pro-antigens that may be useful in the present invention include an Fv, a Fab, and (Fab′)₂ fragments. See, e.g., U.S. Pat. No.4,946,788.

The second domain is a transmembrane (TM) domain, which allows the BAT-CAR to be anchored into the cell membrane of the T cell. The BAT-CAR can be designed to include a transmembrane domain that is attached to the extracellular domain of the CAR. The transmembrane domain may be derived from the same protein or from a different protein from which the other domains of the CAR (e.g., signaling domain, costimulatory domain and hinge domain) are derived. The transmembrane domain may be derived from a natural or from a recombinant source. Where the source is natural, the domain may be derived from any membrane-bound or transmembrane protein. Representative examples of transmembrane domains that may be useful in the present invention include the transmembrane regions of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154.

In some embodiments, the transmembrane domain is attached to the extracellular region of the CAR, e.g., the antigen binding domain of the CAR, via a hinge, such as a hinge from a human protein. Sources of hinge domains include human Ig (immunoglobulin) hinges (e.g., an IgG4 hinge, an IgD hinge), and a CD8 (e.g., CD8a hinge).

The third domain of the BAT-CAR is the T cell activation domain, also known as the intracellular signaling domain, which aids in T cell activation upon binding of the CAR to the tagged protein that is bound to the target cell. An intracellular signaling domain is generally responsible for activation of at least one of the normal effector functions of the effector cell in which the CAR has been introduced. The term “effector function” refers to a specialized function of a cell, which in the case of T cells, includes induction of cytokine and chemokine production, as well as activation of the cytolytic activity of the cells. Examples of intracellular signaling domains for use in the CAR of the invention include the cytoplasmic sequences of the T cell receptor (TCR) and co-receptors that act in concert to initiate signal transduction following antigen receptor engagement. Signals generated through the TCR alone are insufficient for full activation of T cells; thus, a secondary or costimulatory signal is also required. Thus, T cell activation is mediated by two distinct classes of cytoplasmic signaling sequences, namely those that initiate antigen-dependent primary activation through the TCR (i.e., the primary intracellular signaling domains) and those that act in an antigen-independent manner to provide a secondary or costimulatory signal (i.e., the secondary cytoplasmic or costimulatory domain). The primary signaling domain regulates primary activation of the TCR complex either in a stimulatory way, or in an inhibitory way. Primary intracellular signaling domains that act in a stimulatory manner may contain signaling motifs known as immunoreceptor tyrosine-based activation motifs (ITAMs). Representative examples of ITAM-containing primary intracellular signaling domains that may be suitable for use in the present invention include those of CD3ζ, common FcRγ (FCER₁G), Fc-γ RIIa, FcR-β (Fc-ϵ Rib), CD3γ, CD3δ, and CD3 ϵ. In some embodiments, the BAT-CARs include an intracellular signaling domain that contains the primary signaling domain of CD3ζ.

The intracellular signaling domain of the BAT-CAR may also include at least one other intracellular signaling or co-stimulatory domain. A costimulatory molecule is a cell surface molecule other than an antigen receptor or its ligands that is required for an efficient response of lymphocytes to an antigen. Representative examples of co-stimulatory domains that may be useful in the BAT-CARs of the present invention include CD27, CD28, 4-1BB (CD137), OX40, CD30, CD40, PD-1, ICOS, HVEM (LIGHTR), lymphocyte function-associated antigen-1 (LFA-1), CD2, CD7, LIGHT, NKG2C, and B7-H3. CD27 co-stimulation, for example, has been demonstrated to enhance expansion, effector function, and survival of human CART cells in vitro and augments human T cell persistence and antitumor activity in vivo (Song et al. Blood 119(3):696-706 (2012)).

The intracellular signaling domain may be designed to include one or more, e.g., 1, 2, 3, 4, 5, or more, costimulatory signaling domains, which may be linked to each other in a specified or random order, optionally via a linker molecule. Polypeptide linkers that are about 1-10 amino acids in length may join consecutive intracellular signaling sequences. Examples of such linkers include doublets such as Gly-Ser, and single amino acids, e.g., Ala and Gly. Combinations that may constitute the T-cell activation domain may be based on the cytoplasmic regions of CD28, CD137 (4-1BB), OX40 and HVEM, which serve to enhance T cell survival and proliferation; and CD3 CD370 and FcRϵ, which induce T cell activation. For example, CD3ζ, which contains 3 ITAMs, is the most commonly used intracellular domain component of CARs, transmits an activation signal to the T cell after antigen is bound. However, to provide additional co-stimulatory signaling, CD28 and CD137 (4-1BB) domains can be used with CD3ζ which enable the BAT-CAR T cells to transmit the proliferative/survival signals.

A representative example of a polynucleotide that encodes an anti-FL CAR-CD28-4-1BB-CD3 has the sequence designated as SEQ ID NO:1:

(SEQ ID NO: 1)
ATGGCTCTGCCTGTGACAGCTCTGCTGCTGCCTCTGGCTCTGCTTCTGC
ATGCCGCCAGACCTGACGTGGTCATGACACAGACACCTCTGAGCCTGCC
TGTGTCTCTGGGAGATCAGGCCAGCATCAGCTGCAGATCTAGCCAGAGC
CTGGTGCACAGCAACGGCAACACCTACCTGCGGTGGTATCTGCAGAAGC
CCGGCCAGTCTCCTAAGGTGCTGATCTACAAGGTGTCCAACAGAGTGTC
CGGCGTGCCCGATAGATTTTCTGGCAGCGGCTCTGGCACCGACTTCACC
CTGAAGATCAATAGAGTGGAAGCCGAGGACCTGGGCGTGTACTTCTGTA
GCCAGTCTACCCACGTGCCATGGACCTTTGGCGGCGGAACAAAGCTGGA
AATCAAGAGCAGCGCCGACGACGCCAAGAAGGACGCCGCTAAGAAGGAT
GACGCCAAAAAAGACGATGCCAAAAAGGATGGCGGCGTGAAGCTGGACG
AAACAGGCGGAGGACTTGTTCAGCCTGGCGGAGCCATGAAGCTGAGCTG
TGTGACCAGCGGCTTCACCTTCGGCCACTACTGGATGAACTGGGTCCGA
CAGAGCCCTGAGAAAGGCCTGGAATGGGTCGCCCAGTTCAGAAACAAGC
CCTACAACTACGAAACCTACTACAGCGACAGCGTGAAGGGCAGATTCAC
CATCAGCCGGGACGACAGCAAGTCCAGCGTGTACCTGCAGATGAACAAC
CTGCGCGTGGAAGATACCGGCATCTACTACTGTACCGGCGCCAGCTACG
GCATGGAATATCTCGGCCAGGGCACCAGCGTGACCGTGTCTACAACAAC
CCCTGCTCCTCGGCCTCCTACACCAGCTCCTACAATTGCCAGCCAGCCA
CTGTCTCTGAGGCCCGAAGCTTGTAGACCTGCTGCAGGCGGAGCCGTGC
ATACAAGAGGACTGGATTTCGCCTGCGACTTCTGGGTGCTCGTGGTTGT
TGGCGGAGTGCTGGCTTGTTACTCCCTGCTGGTTACCGTGGCCTTCATC
ATCTTTTGGGTCCGAAGCAAGCGGAGCCGGCTGCTGCACAGCGACTACA
TGAACATGACCCCTAGACGGCCCGGACCTACCAGAAAGCACTACCAGCC
TTACGCTCCTCCTAGAGACTTCGCCGCCTACAGATCCAAGCGGGGCAGA
AAGAAGCTGCTGTACATCTTCAAGCAGCCCTTCATGCGGCCCGTGCAGA
CCACACAAGAGGAAGATGGCTGCTCCTGCAGATTCCCCGAGGAAGAAGA
AGGCGGCTGCGAGCTGAGAGTGAAGTTCAGCAGATCCGCCGACGCTCCT
GCCTATCAGCAGGGACAGAACCAGCTGTACAACGAGCTGAACCTGGGGA
GAAGAGAAGAGTACGACGTGCTGGACAAGCGGAGAGGCAGAGATCCTGA
GATGGGCGGAAAGCCCCAGCGGAGAAAGAATCCTCAAGAGGGCCTGTAT
AATGAGCTGCAGAAAGACAAGATGGCCGAGGCCTACAGCGAGATCGGAA
TGAAGGGCGAGCGCAGAAGAGGCAAGGGACACGATGGACTGTACCAGGG
CCTGAGCACCGCCACCAAGGATACCTATGATGCCCTGCACATGCAGGCC
CTGCCACCTAGATGATGA

T cells may be engineered to express BAT-CARs in accordance with known techniques. Generally, a polynucleotide vector is constructed that encodes the BAT-CAR and the vector is transfected into a population of T cells. The cells are then grown under conditions promoting expression of the polynucleotide encoding the BAT-CAR by the T cells. Successful transfection (or transduction which refers to viral-mediated gene integration) and display of BAT-CARs by T cells may be conducted via standard techniques.

In some embodiments, T cells may be engineered to produce BAT-CARs by first constructing a retroviral vector encoding a selected BAT-CAR. Retroviral transduction may be performed using known techniques (e.g., Johnson, et al. Blood 114:535-546 (2009)). The surface expression of BAT-CAR on transduced T cells may be determined, for example, by flow cytometry.

Populations of BAT-CAR T cells may be formulated for administration to a subject using known techniques. Formulations including populations of BAT-CAR-expressing T cells may include one or more pharmaceutically acceptable excipients. Excipients included in the formulations may have different purposes depending, for example, on the nature of the tag-binding domain, the subpopulation of T cells used, and the mode of administration. Representative examples of excipients include saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations including populations of BAT-CAR T cells are typically prepared and cultured in the absence of any non-human components such as animal serum (e.g., bovine serum albumin).

A formulation may include one population of BAT-CAR T cells, or more than one, such as two, three, four, five, six or more populations of BAT-CAR-expressing T cells. The different populations of BAT-CAR T cells may differ in terms of the activation domain, the identity of the subpopulation of T cells, etc.

Systems and Kits of the Invention

Any of the compositions described herein may be comprised in a kit or system. In a non-limiting example, one or more cells for use in cell therapy and/or the reagents to generate one or more cells for use in cell therapy that harbors recombinant expression vectors may be comprised in a kit or system. In some embodiments, a kit includes one or more of the bifunctional compounds disclosed herein. In other embodiments, a kit includes one or more of the bifunctional compounds disclosed herein and one or more reagents (e.g., genetic constructs, delivery vectors) for producing autologous CAR-T cells. In yet other embodiments, a kit includes one or more of the bifunctional compounds disclosed herein and allogeneic CAR-T cells. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly ueful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In particular embodiments, there are one or more apparatuses in the kit suitable for extracting one or more samples from an individual. The apparatus may be a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

Methods

Methods of the present invention include treatment of subjects having or diagnosed with cancer. As used herein, the terms “treat”, “treating”, and “treatment” have their ordinary and customary meanings, and include one or more of blocking, ameliorating, or decreasing in severity and/or frequency a symptom of cancer in a subject. In some embodiments, the subject receiving treatment is a human. In other embodiments, the subject is a non-human animal, e.g., a non-human primate, bird, horse, cow, goat, sheep, a companion animal, such as a dog, cat or rodent, or other mammal.

Cancers that may be amenable to treatment with the treatment modalities of the present invention are characterized by presence of solid tumors. Broadly, they include adenomas, carcinomas, sarcomas, lymphomas, both adult and pediatric alike. The cancers may be vascularized, or not yet substantially vascularized, or non-vascularized tumors.

Representative examples of cancers characterized by solid tumors which may be treated in accordance with the present invention include breast (including HER₂+ and metastatic), colorectal, colon, esophageal, bile duct, lung (including small cell and non-small cell lung tumors, adenocarcinoma of the lung and squamous carcinoma of the lung), liver, epidermoid tumors, squamous tumors such as head and neck tumors, epithelial squamous cell cancer, thyroid, cervical, ovarian, neuroendocrine tumors of the digestive system, neuroendocrine tumors, pheochromacytomas, cancer of the peritoneum, hepatoblastoma, HPCR, brain cancers (e.g., diffuse intrinsic pontine glioma, capillary hemangioblastomas, meningiomas, and cerebral metastases, gliomas, glioblastomas (glioblastoma multiforme) and neuroblastomas, and medulloblastoma, ependymoma), bladder cancer, hepatoma, endometrial or uterine carcinoma, salivary gland carcinoma, kidney or renal cancer, bone cancer, soft tissue sarcoma (including embryonal and alveolar rhabdomyosarcoma, GIST, rectal, pancreatic, prostate, gastrointestinal (gastric and stomach), alveolar soft part sarcoma and clear cell sarcoma), cholangiocarcinoma, bile cancer, gallbladder carcinoma, myeloma, vulval cancer, penile carcinoma, retinal, androgen-dependent tumors, androgen-independent tumors, Kaposi's sarcoma, synovial sarcoma, vasoactive intestinal peptide secreting tumor, CNS neoplasms, melanoma, rhabdomyosarcoma, including, EMB, RMS, ALV, Wilm's cancer, Ewing's cancer, osteosarcoma, PNT, rhabdoid, rhabdomyosarcoma, retinoblastoma, adrenal cortical cancer, adrenal cancer, and leiomyosarcoma.

Representative examples of light-accessible tumors include those found in the tissues of skin, cervix, bladder, prostate, bile duct, pancreas, stomach, brain, mouth, larynx, vagina, vulva and nasal passages. Light may be delivered to some tumors via interstitial therapy, which involves using imaging tests (such as CT scans) to guide fiber optics directly into tumors using needles or other minimally invasive means. Light delivered via interstitial therapy can be used to treat tumors found in the breast, ovaries, head and neck, prostate, liver and lungs. For tumors at greater depths within a patient's body, light can be delivered to tumor tissue during or after surgery to target any cancerous tissues that may be remaining after tumor excision.

Cancers to be treated include primary tumors and secondary or metastatic tumors e.g., metastasized from lung, breast, or prostate, as well as recurrent or refractory tumors. Recurrent tumors encompass tumors that appear to be inhibited by treatment with such agents, but which recur up to five years, or even up to ten years, or longer, after treatment is discontinued. Refractory tumors are tumors that were unresponsive or resistant to treatment with one or more conventional, approved or experimental therapies for the particular tumor type.

The therapeutic methods of the present invention may be “first-line”, i.e., an initial treatment in patients who not yet undergone any anti-cancer treatment, either alone or in combination with other treatments. The therapeutic methods of the present invention may also be “second-line” in the sense that they are administered to patients who have undergone at least one prior anti-cancer treatment regimen, e.g., chemotherapy, radioimmunotherapy, toxin therapy, prodrug-activating enzyme therapy, antibody therapy, surgical therapy, immunotherapy, radiation therapy, targeted therapy or any combination thereof either alone or in combination with other treatments. In some cases, the prior therapy may have been unsuccessful or partially successful but where the patient became intolerant to the particular treatment. Methods of the present invention may also be used as an adjuvant treatment, e.g., to inhibit reoccurrence of cancer in patients with no currently detectable disease or after surgical removal of tumor.

The formulation contains the BAT-CAR T cells in a number that is effective for the treatment of the specific cancer. Thus, therapeutically-effective populations of BAT-CAR T cells are administered to subjects. The number of BAT-CART cells administered to a subject will vary between wide limits, depending upon the location, source, identity, extent and severity of the cancer, the age and condition of the individual to be treated, etc. A physician will ultimately determine appropriate dosages to be used. In general, formulations are administered that contain from about 1×10⁴ to about 1×10¹⁰ BAT-CAR T cells. In some embodiments, the formulation contains from about 1×10⁵ to about 1×10⁹ BAT-CAR T cells, from about 5×10⁵ to about 5×10⁸ BAT-CAR T cells, or from about 1×10⁶ to about 1×10⁷ BAT-CART cells.

The formulation of BAT-CAR T cells may be administered to a subject in need thereof in accordance with acceptable medical practice. An exemplary mode of administration is intravenous injection. Other modes include intratumoral, intradermal, subcutaneous (s.c., s.q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of the formulations can be used to effect such modes of administration.

The “compounds” and the BAT-CAR T cells are co-administered to the subject, which for purposes of the present invention includes administration during the same treatment regimen. The compounds may be administered to a subject prior to, or concurrent with, or after administration of the BAT-CAR T cells, such that the compounds will bind the target cells and that once unmasked, the BAT-CAR cells will bind the unmasked pro-antigen or tag.

Formulations containing the compounds may be administered to a subject in an amount which is effective for treating the specific cancer. The compounds may be formulated for administration to a subject using techniques known to the skilled artisan. Formulations of the compounds may include a pharmaceutically acceptable excipient, which may be selected based on factors such as the nature of the targeting moiety, the pro-antigen, and the mode of administration. Representative examples of generally used excipients include saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

In general, the amount of the compound in the formulation administered to a subject will vary between wide limits, depending upon the location, source, identity, extent and severity of the cancer, the age and condition of the individual to be treated, etc. A physician will ultimately determine appropriate dosages to be used. Typically, formulations may contain from about 0.1 mg/kg to about 100 mg/kg body weight of the compound, and in some embodiments from about 1 mg/kg to about 10 mg/kg body weight of the compound, taking into account the routes of administration, symptoms, etc. Generally, the dosage of a compound of the present application administered to a subject to treat a disease or disorder such as cancer is in the range of 0.01 to 500 mg/kg, e.g., in the range of 0.1 mg/kg to 100 mg/kg, of the subject's body weight. For example, the dosage of compound administered to a subject may be in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a subject's body weight. [000141] The compounds may be administered to a subject in need thereof in accordance with acceptable medical practice. An exemplary mode of administration is intravenous injection. Other modes include intratumoral, intradermal, subcutaneous (s.c., s.q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of the formulations can be used to effect administration of the compound.

Activation of the pro-antigen is achieved by localized administration of light of an appropriate wavelength to effect cleavage or removal of the protection group from the tag or recognition domain. The nature of the protection group will determine the appropriate wavelength of light to be administered. The photosensitive protection groups of the present invention are cleaved upon exposure to UV irradiation or MALDI (Matrix-Assisted Laser Desorption/Ionization) conditions. Generally, the wavelength of light administered to a subject to activate the CAR-T cells by cleaving the protection group from the pro-antigen to produce the tag or recognition domain is in the range of 10-600 nm, e.g., in the range of 250-550 nm. For example, the wavelength of light administered to a subject may be in the range of 250-300 nm, 300-350 nm, 350-400 nm, 400-450 nm, 450-500 nm or 500-550 nm. In another example, the wavelength of light administered to a subject to cleave the photosensitive protection groups is 550 nm or less, preferably 525 nm or less, 500 nm or less, 475 nm or less, 450 nm or less, 425 nm or less, 400 nm or less, 375 nm or less, 350 nm or less, 325 nm or less, 300 nm or less, 275 nm or less, 250 nm or less, 225 nm or less or 200 nm or less.

Administration frequencies of formulations containing populations of BAT-CAR-T cells and formulations of the compounds, as well as both the frequency and duration of exposure to the appropriate wavelengths of light will vary depending on factors that may include the disease being treated, the structure of the BAT-CAR-T cells and the compounds, and the modes of administration. Each may be independently administered 4, 3, 2 or once daily, every other day, every third day, every fourth day, every fifth day, every sixth day, once weekly, every eight days, every nine days, every ten days, bi-weekly, monthly and bi-monthly. The duration of treatment will also vary, and be based for example, on the disease being treated and will be best determined by the attending physician. However, continuation of treatment is contemplated to last for a number of days, weeks, or even months.

The methods of the present application may entail independent or co-administration of the compounds, the BAT-CAR-T cells and the appropriate wavelength of light to the subject in a single, one-time dose, or in multiple doses (e.g., 2, 3, 4, 5, 6, 7, 8, 10, 15, 20, or more doses). Thus, BAT-CAR-T cells may be administered in a single dose, while the frequency of co-administration of the compounds and appropriate wavelength of light may range from once every day to a single dose. In another example, the compounds, the BAT-CAR-T cells and the appropriate wavelength of light may be co-administered from a single dose up to about once a week up to about once every six weeks. In some embodiments, the administration may be a combination of single and co-administrations (e.g. the compounds, the BAT-CAR-T cells and the appropriate wavelength of light may be co-administered for the first dose, and then administration of the compounds and appropriate wavelength of light once every three weeks up to about once every 4-6 weeks).

Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies. In the context of the present invention, it is contemplated that cell therapy could be used similarly in conjunction with chemotherapeutic, radiotherapeutic, or immunotherapeutic intervention, as well as pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

Chemotherapy

Cancer therapies also include a variety of combination therapies with both chemical and radiation based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the invention, for example before, during and/or after administration of the invention.

Radiotherapy

Other factors that cause DNA damage and have been used extensively include what are commonly known as gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include PD-1, PD-L1, CTLA4, carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs'surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR₄ or DR₅/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

These and other aspects of the present invention will be further appreciated upon consideration of the following working example, which is intended to illustrate certain particular embodiments of the invention, but which is not intended to limit its scope, as defined by the claims.

### EXAMPLES

### Example 1

### Construction of Anti-Fluorescein CART T and its Components

Construct #1, encoding a polypeptide comprising “anti-Fluorescein CAR T”

anti-Fluorescein-4M5.3 antibody (antigen binding
domain)
(SEQ ID NO: 2)
DVVMTQTPLSLPVSLGDQASISCRSSQSLVHSNGNTYLRWYLQKPGQSP
KVLIYKVSNRVSGVPDRFSGSGSGTDFTLKINRVEAEDLGVYFCSQSTH
VPWTFGGGTKLEIKSSADDAKKDAAKKDDAKKDDAKKDGGVKLDETGGG
LVQPGGAMKLSCVTSGFTFGHYWMNWVRQSPEKGLEWVAQFRNKPYNYE
TYYSDSVKGRFTISRDDSKSSVYLQMNNLRVEDTGIYYCTGASYGMEYL
GQGTSVTVS
Signal peptide-CD8a-sp|P01732|1-21
(SEQ ID NO: 3)
MALPVTALLLPLALLLHAARP
Hinge region-CD8a-sp|P01732|138-182
(SEQ ID NO: 4)
TTTPAPRPPTPAPTIASQPLSLRPEACRPAAGGAVHTRGLDFACD
Transmembrane region (TM)-CD28-sp|P10747|153-179
(SEQ ID NO: 5)
FWVLVVVGGVLACYSLLVTVAFIIFWV
Intracellular domain (ICD)-CD28-sp|P10747|180-220
(SEQ ID NO: 6)
RSKRSRLLHSDYMNMTPRRPGPTRKHYQPYAPPRDFAAYRS
Intracellular domain (ICD)-41BB-sp|Q07011|214-255
(SEQ ID NO: 7)
KRGRKKLLYIFKQPFMRPVQTTQEEDGCSCRFPEEEEGGCEL
Intracellular domain (ICD)-CD3z-sp|P20963|52-164
(SEQ ID NO: 8)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR
anti-Fluorescein CAR T-full peptide
(SEQ ID NO: 9)
MALPVTALLLPLALLLHAARPDVVMTQTPLSLPVSLGDQASISCRSSQS
LVHSNGNTYLRWYLQKPGQSPKVLIYKVSNRVSGVPDRFSGSGSGTDFT
LKINRVEAEDLGVYFCSQSTHVPWTFGGGTKLEIKSSADDAKKDAAKKD
DAKKDDAKKDGGVKLDETGGGLVQPGGAMKLSCVTSGFTFGHYWMNWVR
QSPEKGLEWVAQFRNKPYNYETYYSDSVKGRFTISRDDSKSSVYLQMNN
LRVEDTGIYYCTGASYGMEYLGQGTSVTVSTTTPAPRPPTPAPTIASQP
LSLRPEACRPAAGGAVHTRGLDFACDFWVLVVVGGVLACYSLLVTVAFI
IFWVRSKRSRLLHSDYMNMTPRRPGPTRKHYQPYAPPRDFAAYRSKRGR
KKLLYIFKQPFMRPVQTTQEEDGCSCRFPEEEEGGCELRVKFSRSADAP
AYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQA
LPPR**

### Example 2

### Construction of Anti-Anthraquinone-2-Carboxylate CART T and its Components

Construct #2, encoding a polypeptide comprising “anti-Anthraquinone-2-carboxylate CAR T”

anti-Anthraquinone-2-carboxylate-MC48.B11
antibody (antigen binding domain)
(SEQ ID NO: 10)
QVRLQGSGPSLVKPSQTLSLTCTVSGFSLTSNAVDWVRQAPGKVPEWLG
FIRGGGSTFYNSALKSRLSITRDTSKSQVSLSLSSVTTEDTAVYYCARA
SCSGDIYTDTCGIDYWGPGLLVTVSSEGKSSGSGSESKVDQSALTQPSS
VSRSLGQSVSITCSGSSSNVGAGNYVNWFRLIPGSAPKSLIYAATTRAS
GVPDRFSGSRSGNTATLTISSLQAEDEADYYCSSYDITAVNLFGSGTRL
TVLG
Signal peptide-CD8a-sp|P01732|1-21
(SEQ ID NO: 3)
MALPVTALLLPLALLLHAARP
Hinge region-CD8a-sp|P01732|138-182
(SEQ ID NO: 4)
TTTPAPRPPTPAPTIASQPLSLRPEACRPAAGGAVHTRGLDFACD
Transmembrane region (TM)-CD28-sp|P10747|153-179
(SEQ ID NO: 5)
FWVLVVVGGVLACYSLLVTVAFIIFWV
Intracellular domain (ICD)-CD28-sp|P10747|180-220
(SEQ ID NO: 6)
RSKRSRLLHSDYMNMTPRRPGPTRKHYQPYAPPRDFAAYRS
Intracellular domain (ICD)-41BB-sp|Q07011|214-255
(SEQ ID NO: 7)
KRGRKKLLYIFKQPFMRPVQTTQEEDGCSCRFPEEEEGGCEL
Intracellular domain (ICD)-CD3z-sp|P20963|52-164
(SEQ ID NO: 8)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR
anti-Anthraquinone-2-carboxylate CAR T-full
peptide
(SEQ ID NO: 11)
MALPVTALLLPLALLLHAARPQVRLQGSGPSLVKPSQTLSLTCTVSGFS
LTSNAVDWVRQAPGKVPEWLGFIRGGGSTFYNSALKSRLSITRDTSKSQ
VSLSLSSVTTEDTAVYYCARASCSGDIYTDTCGIDYWGPGLLVTVSSEG
KSSGSGSESKVDQSALTQPSSVSRSLGQSVSITCSGSSSNVGAGNYVNW
FRLIPGSAPKSLIYAATTRASGVPDRFSGSRSGNTATLTISSLQAEDEA
DYYCSSYDITAVNLFGSGTRLTVLGTTTPAPRPPTPAPTIASQPLSLRP
EACRPAAGGAVHTRGLDFACDFWVLVVVGGVLACYSLLVTVAFIIFWVR
SKRSRLLHSDYMNMTPRRPGPTRKHYQPYAPPRDFAAYRSKRGRKKLLY
IFKQPFMRPVQTTQEEDGCSCRFPEEEEGGCELRVKFSRSADAPAYQQG
QNQLYNELNLGRREEYDVLDKRRGRDPEMGGKPQRRKNPQEGLYNELQK
DKMAEAYSEIGMKGERRRGKGHDGLYQGLSTATKDTYDALHMQALPPR**

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound comprising a pro-antigen covalently linked to a targeting moiety, wherein the pro-antigen comprises a small molecule having a photocleavable protecting group and the targeting moiety specifically binds a tumor associated antigen.

2. The compound of claim 1, wherein the small molecule is a fluorescent molecule.

3. The compound of claim 2, wherein the fluorescent molecule is a fluorescein, an anthracene, an alexa fluor, a rhodamine, a rhodol, an acridine or a xanthene.

4. The compound of claim 1, wherein the photocleavable protecting group is an ortho-nitrobenzyl group, a phenacyl ester group, an 8-quinolinyl benzenesulfonate group, a dicoumarin group, a 6-bromo-7-alkoxycoumarin-4-ylmethoxycarbonyl group, a bimane group or a bis-arylhydrazone group.

5. The compound of claim 4, wherein the photocleavable protecting group is an ortho-nitrobenzyl group.

6. The compound of claim 1, represented by formula (A) or (A′): 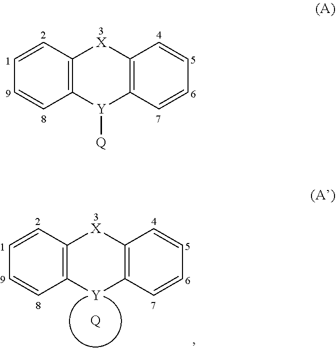
wherein X is C or O, Y is C or N, the photocleavable protecting group is present at one or more of positions 1-9 and Q represents one or more optionally substituted rings or a photocleavable protecting group.

7. The compound of claim 6, wherein the optionally substituted rings are saturated or non-saturated 4-7 member carbocyclic or heterocyclic rings or a fused ring system, wherein the heteroatoms are N, O or S.

8. The compound of claim 6, represented by formula (I): 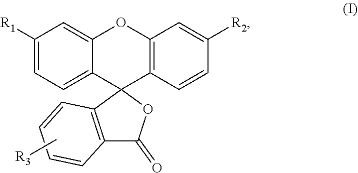
wherein R₁ is O, OH or the photocleavable protecting group,
R₂ is O, OH or the photocleavable protecting group; and
R₃ is 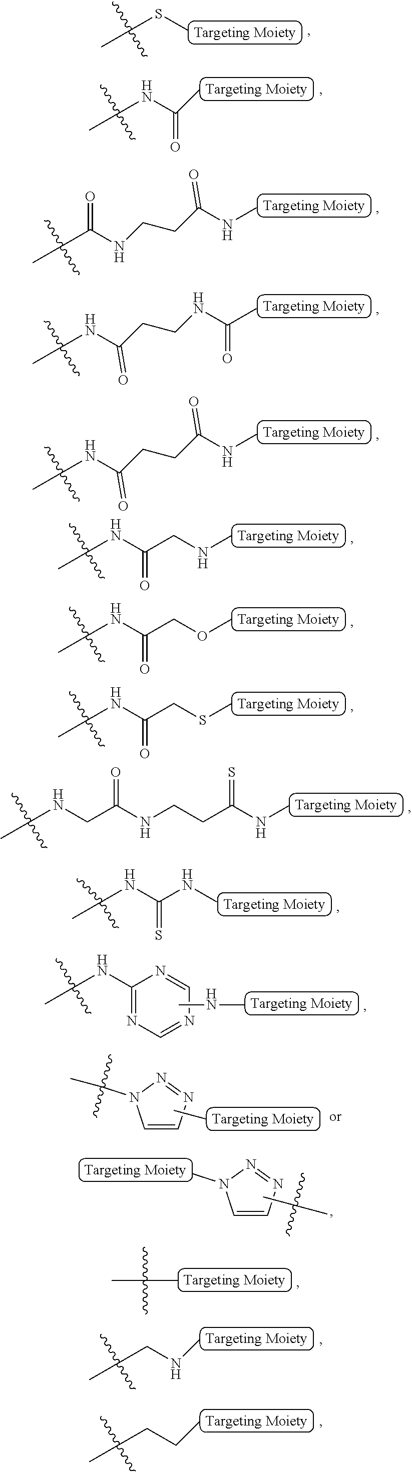 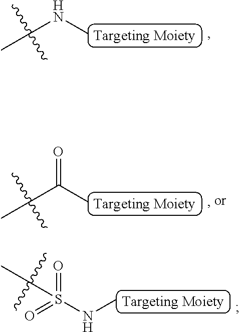
or a stereoisomer thereof.

9. The compound of claim 6, wherein the photocleavable protecting group is ortho-nitrobenzyl group, a phenacyl ester group, an 8-quinolinyl benzenesulfonate group, a dicoumarin group, a 6-bromo-7-alkoxycoumarin-4-ylmethoxycarbonyl group, a bimane group or a bis-arylhydrazone group.

10. The compound of claim 9, wherein the photocleavable protecting group is an ortho-nitrobenzyl group.

11. The compound of claim 10, wherein the ortho-nitrobenzyl group is 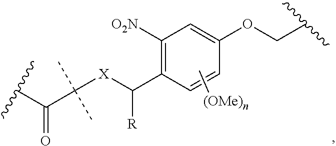
wherein X is NH or O, R is C1-4 alkyl or H and n is 0-3.

12. The compound of claim 10, wherein the ortho-nitrobenzyl group is

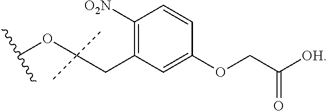

13. The compound of claim 12, represented by formula (Ia): 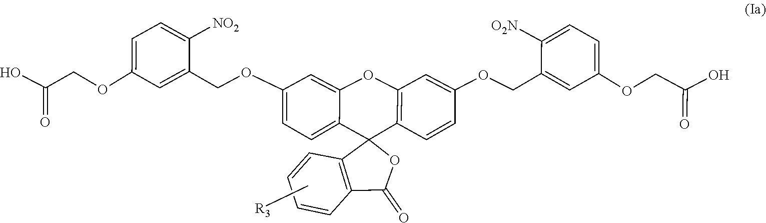
or a stereoisomer thereof.

14. The compound of claim 13, represented by formula (Ib): 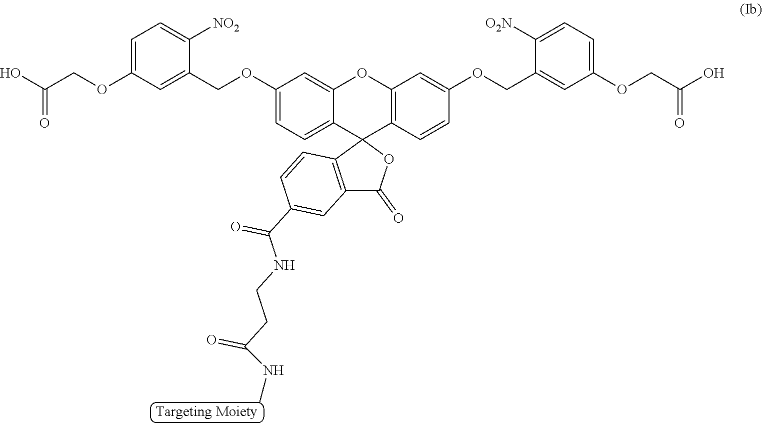 [SMILES: CNC(=O)CCNC(=O)c1ccc2c(c1)C(=O)OC21c2ccc(OCc3cc(OCC(=O)O)ccc3[N+](=O)[O-])cc2Oc2cc(OCc3cc(OCC(=O)O)ccc3[N+](=O)[O-])ccc21]
or a stereoisomer thereof.

15. The compound of claim 6, represented by formula (II): 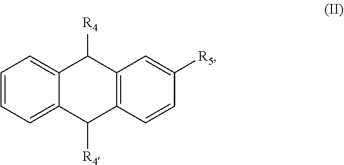
wherein each of R₄ and R₄′ is independently O or a photocleavable protecting group; and
R₅ is 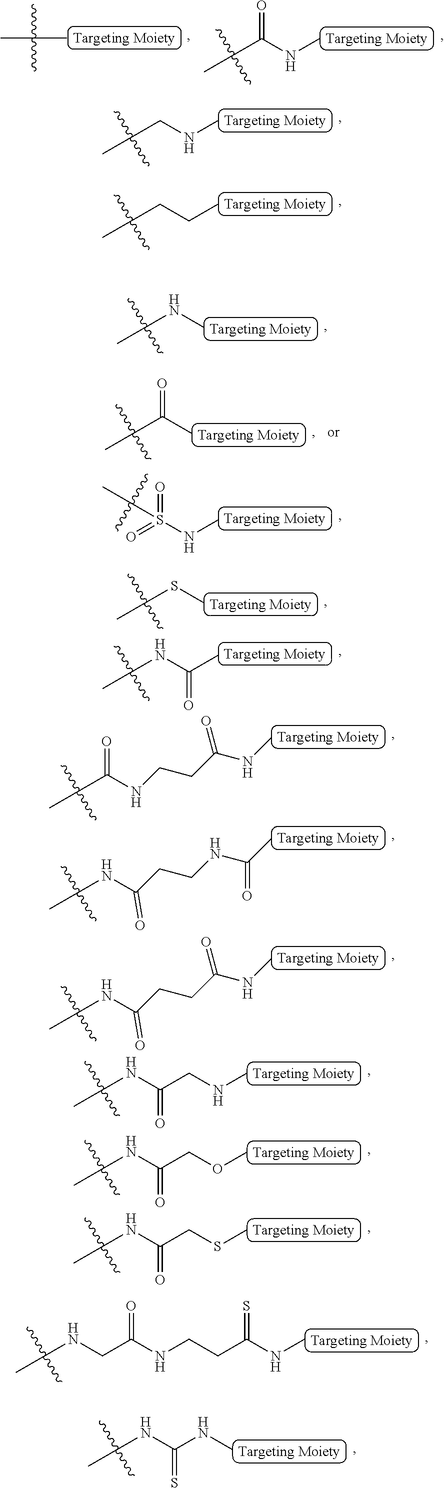 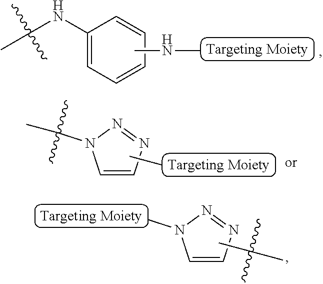
or a stereoisomer thereof.

16. The compound of claim 15, wherein the photocleavable protecting group is an ortho-nitrobenzyl group, a phenacyl ester group, an 8-quinolinyl benzenesulfonate group, a dicoumarin group, a 6-bromo-7-alkoxycoumarin-4-ylmethoxycarbonyl group, a bimane group or a bis-arylhydrazone group.

17. The compound of claim 16, wherein the photocleavable protecting group is an ortho-nitrobenzyl group.

18. The compound of claim 17, wherein the ortho-nitrobenzyl group is 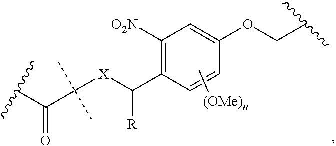
wherein X is NH or O, R is C1-4 alkyl or H and n is 0-3.

19. The compound of claim 17, wherein the ortho-nitrobenzyl group is

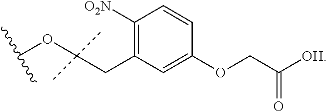

20. The compound of claim 19, represented by formula (IIa): 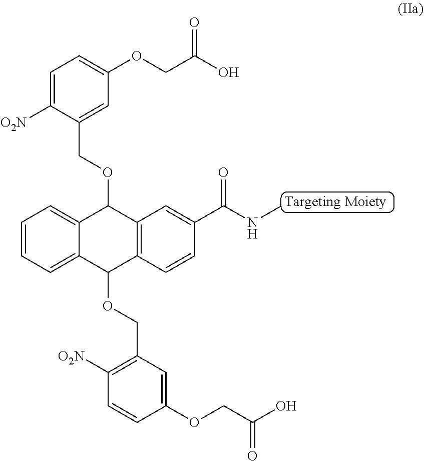 [SMILES: CNC(=O)c1ccc2c(c1)C(OCc1cc(OCC(=O)O)ccc1[N+](=O)[O-])c1ccccc1C2OCc1cc(OCC(=O)O)ccc1[N+](=O)[O-]]
or a stereoisomer thereof.

21. The compound of claim 1, wherein the targeting moiety comprises an antibody, an antibody fragment, a ligand, an aptamer or a nanobody.

22. The compound of claim 1, wherein the targeting moiety specifically binds a tumor associated antigen selected from the group consisting of platelet derived growth factor receptor alpha (PDGFRa), activin a receptor type 1 (ACVR₁), human epidermal growth factor receptor 2 (Her2), prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, an abnormal p53 protein, mesothelin, EGFRvIII, EGFR₁, diganglioside GD2, interleukin 13 receptor α (IL13Rα), fibroblast activation protein (FAP), and L1 cell adhesion molecule (L1CAM).

23. The compound of claim 1, wherein the targeting moiety specifically binds a tumor associated antigen expressed or overexpressed on a light-accessible tumor.

24. The compound of claim 23, wherein the light-accessible tumor is located in tissues of breasts, ovaries, skin, cervix, bladder, prostate, bile duct, pancreas, stomach, brain, mouth, larynx, vagina, vulva or nasal passages.

25. The compound of claim 1, wherein the targeting moiety is selected from the group consisting of trastuzumab, cetuximab, panitumamab, zalutumumab, nimotuzumab, matuzumab, gefitinib, erlotinib, lapatinib, pertuzumab, tositumomab, rituximab, ibritumomab tiuxetan, daclizumab, CEA-scan, colo101, OC125 monoclonal antibody, Ab75705, anti-AFP antibody or fragment thereof, humanized B3, B72.3, bevacizumab, anti-CD99 antibody or fragment thereof, anti-HER₂ antibody or fragment thereof and anti-EGFR antibody or fragment thereof

26. A pharmaceutical composition comprising a therapeutically effective amount of the compound of claim 1, and a pharmaceutically acceptable carrier.

27. A system comprising:
a) a compound of claim 1; and
b) CAR-T cells that specifically recognize an unmasked compound of claim 1.

28. A kit comprising:
a) a compound of claim 1.

29. The kit of claim 28 further comprising:
b) reagents for producing autologous CAR-T cells that specifically recognize an unmasked compound of claim 1.

30. The kit of claim 28 further comprising:
c) allogeneic CAR-T cells that specifically recognize an unmasked compound of claim 1.

31. A method of treating cancer comprising administering to a subject in need thereof, a therapeutically effective amount of the compound of claim 1, light at a wavelength appropriate to cleave the protecting group, and one or more chimeric antigen receptor T (CAR-T) cells, wherein the CAR-T cells comprise an extracellular ligand that specifically binds unmasked pro-antigen.

32. The method of claim 31, wherein the compound and light are administered to the subject prior to administration of the CAR-T cells.

33. The method of claim 31, wherein the compound and light are administered to the subject after administration of the CAR-T cells.

34. The method of claim 31, wherein the compound and light are administered to the subject concomitantly with administration of the CAR-T cells.

35. The method of claim 31, wherein the compound is administered at a dose of 0.01 mg/kg to 500 mg/kg body weight.

36. The method of claim 31, wherein the CAR-T cells are administered at a dose of 10⁴ to 10⁹ cells per kg body weight.

37. The method of claim 31, wherein the light is administered at a wavelength of 10 to 600 nm.

38. The method of claim 31, wherein the compound and the CAR-T cells are administered parenterally.

39. The method of claim 31, wherein the light is administered via a non-invasive procedure or a minimally-invasive procedure.

40. The method of claim 31, wherein the light is administered during or after surgery.

41. The method of claim 31, wherein the compound and the light are administered more than once and the CAR-T cells are administered once.

42. The method of claim 31, wherein the light is administered to the breasts, ovaries, skin, cervix, bladder, prostate, bile duct, pancreas, stomach, brain, mouth, larynx, vagina, vulva or nasal passages.
